(19) United States
(12) Patent Application Publication
(10) Pub. No.: US 2025/0197938 A1
(43) Pub. Date: Jun. 19, 2025

(54) SPATIAL ANALYSIS OF GENETIC VARIANTS

(71) Applicant: 10x Genomics, Inc., Pleasanton, CA (US)

(72) Inventor: Anton Jan Bjorninen, Stockholm (SE)

(21) Appl. No.: 18/918,798

(22) Filed: Oct. 17, 2024

Related U.S. Application Data
(60) Provisional application No. 63/545,122, filed on Oct. 20, 2023.

(51) Int. Cl. C12Q 1/6876; C12Q 1/44; C12Q 1/48; C12Q 1/6837; C12Q 1/6841; C12Q 1/6869

(52) U.S. Cl. CPC C12Q 1/6876; C12Q 1/44; C12Q 1/48; C12Q 1/6837; C12Q 1/6841; C12Q 1/6869

(57) ABSTRACT

Provided herein are methods of identifying genetic variants in biological samples. In particular, the present disclosure uses spatial transcriptomic templated ligation methods and compositions to identify the presence or absence, and the location of genetic variants in the transcriptome or genome of a biological sample.

### CROSS-REFERENCE TO RELATED APPLICATIONS

This application claims the benefit of U.S. Provisional Application No. 63/545,122 filed Oct. 20, 2023, which is herein incorporated by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide substantial analyte data for dissociated tissue (i.e., single cells), but fail to provide information regarding the position of the single cell in a parent biological sample (e.g., tissue sample).

Spatial analysis of RNA in fixed tissue using in situ ligation of templated probe pairs has previously been demonstrated. See, e.g., Credle et al., Nucleic Acids Research, Volume 45, Issue 14, 21 August 2017, Page e128 (2017). Current approaches like in situ ligation used to identify single nucleotide polymorphisms and mutations in a nucleic acid can suffer from poor specificity and/or sensitivity. Improved methods for analyzing nucleic acids present in and/or from a biological sample with increased specificity and sensitivity are therefore needed. The present disclosure addresses this and other needs.

### SUMMARY

This disclosure is predicated on the discovery that templated probe pairs may hybridize to sequences without 100% complementarity. For instance, a probe may hybridize to a wild-type sequence with 100% complementarity, but may also hybridize to a sequence having one change or mismatch compared to the wild-type sequence (e.g., having less than 100% complementarity). In these instances, deciphering the difference between the wild-type and mutated nucleic acid sequences using existing methods of templated ligation may be challenging. The present disclosure addresses this issue, and provides methods of spatial analysis with identification of genetic variants, such as single nucleotide polymorphisms (“SNPs”) or other mutations, in a target nucleic acid. The methods utilize templated ligation of multiple probes (e.g., two probes) that hybridize to adjacent or near-adjacent sequences on a target nucleic acid. Hybridized probes may be distinguished based on level of complementarity to the target nucleic acid. That is, probes that hybridize to the target nucleic acid with less than 100% complementarity may be digested or nicked, thereby forgoing downstream processing and analysis the probes, whereas probes that hybridize to a target nucleic acid with 100% complementarity can avoid digestion or nicking and may thereby undergo further processing and analysis. The undigested and unnicked probes can be analyzed for identification of the SNP or mutation in the target nucleic acid.

Thus, in one aspect, disclosed herein is a method for spatially tagging a target nucleic acid in a biological sample, the method comprising: (a) contacting a plurality of first probes and a plurality of second probes with the biological sample on a first substrate, wherein the biological sample comprises a plurality of target nucleic acids, each target nucleic acid comprising either a wild-type sequence or a genetic variant sequence, wherein each probe of the plurality of first probes and each probe of the plurality of second probes comprises a sequence complementary to a target nucleic acid of the plurality of target nucleic acids, and wherein each probe of the plurality of first probes or each probe of the plurality of second probes comprises a capture probe capture domain that is complementary to a capture domain; (b) hybridizing the plurality of first probes and the plurality of second probes to the plurality of target nucleic acids, thereby generating hybridized first and second probes, wherein the plurality of first probes and the plurality of second probes are configured to hybridize to both the wild-type sequence and the genetic variant sequence; (c) contacting one or more blocking probes with the hybridized first and second probes, wherein the one or more blocking probes are configured to hybridize to the hybridized first and second probes at regions that are not hybridized to the target nucleic acid, thereby generating protected first and second probes; (d) contacting an endonuclease with the protected first and second probes, wherein the endonuclease cleaves protected first or second probes that are hybridized to target nucleic acids and include a mismatch; (c) generating ligation products by ligating the protected first and second probes; (f) releasing the ligation products from the target nucleic acids; (g) releasing the one or more blocking probes from the ligation products; and (h) hybridizing the ligation products to a plurality of capture probes affixed to an array, wherein each capture probe of the plurality of capture probes comprises: (i) a spatial barcode and (ii) the capture domain, thereby spatially tagging the target nucleic acid in the biological sample.

In another aspect, disclosed herein is a method for spatially tagging a target nucleic acid in a biological sample, the method comprising: (a) contacting a plurality of first probes and a plurality of second probes with the biological sample on a first substrate, wherein the biological sample comprises a plurality of target nucleic acids, each target nucleic acid comprising either a wild-type sequence or a genetic variant sequence, wherein each probe of the plurality of first probes and each probe of the plurality of second probes comprises a sequence complementary to a target nucleic acid of the plurality of target nucleic acids, and wherein each probe of the plurality of first probes or each probe of the plurality of second probes comprises a capture probe capture domain that is complementary to a capture domain; (b) hybridizing the plurality of first probes and the plurality of second probes to the plurality of target nucleic acids, thereby generating hybridized first and second probes, wherein the plurality of first probes and the plurality of second probes are configured to hybridize to both the wild-type sequence and the genetic variant sequence; (c) generating ligation products by ligating the hybridized first and second probes; (d) contacting one or more blocking probes with the ligation products, wherein the one or more blocking probes are configured to hybridize to the hybridized ligation products at regions that are not hybridized to the target nucleic acid, thereby generating protected ligation products; (e) contacting an endonuclease with the protected ligation products, wherein the endonuclease cleaves protected ligation products that are hybridized to target nucleic acids and include a mismatch; (f) releasing the ligation products from the target nucleic acids; (g) releasing the one or more blocking probes from the ligation products; and (h) hybridizing the ligation products to a plurality of capture probes affixed to an array, wherein each capture probe of the plurality of capture probes comprises: (i) a spatial barcode and (ii) the capture domain, thereby spatially tagging the target nucleic acid in the biological sample.

In some instances, a probe of the plurality of first probes comprises a sequence complementary to the genetic variant sequence. In some instances, a probe of the plurality of second probes comprises a sequence complementary to the genetic variant sequence. In some instances, n each probe of the plurality of first probes comprises one of four different nucleotide sequences within a region configured to hybridize to the target nucleic acid. In some instances, each probe of the plurality of second probes comprises one of four different nucleotide sequences within a region configured to hybridize to the target nucleic acid. In some instances, each probe of the plurality of first probes comprises identical sequences and/or each probe of the plurality of second probes comprises identical sequences.

In some instances, the endonuclease generates a nick in the protected first and/or second probes or the protected ligation products. In some instances, the endonuclease cleaves the protected first and/or second probes or the protected ligation products that are hybridized to the genetic variant sequence that includes the mismatch. In some instances, the endonuclease cleaves the protected first and/or second probes or the protected ligation products that are hybridized to the wild-type sequence that includes the mismatch. In some instances, the endonuclease is an S1 nuclease. In some instances, the regions of the hybridized first and second probes that are not hybridized to the target nucleic acid comprises 5′ single-stranded ends of the hybridized first probes and/or 3′ single-stranded ends of the hybridized second probes. In some instances, the one or more blocking probes comprises one or more sequences complementary to the 5′ single-stranded ends of the hybridized first probes and/or the 3′ single-stranded ends of the hybridized second probes. In some instances, the one or more blocking probes comprise a poly(T) sequence. In some instances, one or more blocking probes comprise a hairpin sequence located at 5′ ends of the one or more blocking probes. In some instances, a blocking probe of the one or more blocking probes and a first probe of the plurality of first probes are on one contiguous nucleic acid sequence. In some instances, a blocking probe of the one or more blocking probes and a second probe of the plurality of second probes are on one contiguous nucleic acid sequence. In some instances, the hairpin sequence comprises a cleavable linker selected from the group consisting of a photocleavable linker, UV-cleavable linker, and an enzyme-cleavable linker. In some instances, the hairpin sequence comprises a target recognition sequence for a restriction endonuclease or an endoribonuclease.

In some instances, the releasing the one or more blocking probes comprises contacting the one or more blocking probes with a restriction endonuclease or an endoribonuclease. In some instances, the releasing the one or more blocking probes comprises heating the one or more blocking probes. In some instances, the releasing the one or more blocking probes comprises contacting the one or more blocking probes with a uracil-DNA glycosylase and a second endonuclease. In some instances, the second endonuclease comprises Endonuclease VIII, Endonuclease III, Endonuclease V, UNG1, UNG2, SMUG1, thymine-DNA glycosylase (TDG), or methyl CpG binding domain-4 (MBD4).

In some instances, probes of the plurality of first probes or the plurality of second probes comprises a plurality of caged nucleotides, wherein a caged nucleotide of the plurality of caged nucleotides comprises a caged moiety that blocks the capture probe capture domain from hybridizing to the capture domain of the capture probe on the array.

In some instances, the methods also include releasing the caged moiety from the capture probe capture domain, thereby allowing the capture probe capture domain to hybridize to the capture domain of the capture probe on the array. In some instances, the releasing comprises photolysis of the caged moiety. In some instances, the releasing comprises exposing the caged moiety to light pulses, wherein the light pulses have a wavelength of about or less than 360 nm. In some instances, the caged moiety is selected from the group consisting of 6-nitropiperonyloxymethyl (NPOM), 1-(ortho-nitrophenyl)-ethyl (NPE), 2-(ortho-nitrophenyl)propyl (NPP), 7-(diethylamino)-4-(hydroxymethyl)-coumarin (DEACM), and nitrodibenzofuran (NDBF). In some instances, the caged nucleotide comprises a non-naturally-occurring nucleotide selected from the group consisting of NPOM-caged adenosine, NPOM-caged guanosine, NPOM-caged uridine, and NPOM-caged thymidine.

In some instances, generating the ligation products comprises ligating hybridized first and second probes by enzymatic ligation, wherein the enzymatic ligation is performed using a ligase. In some instances, the ligase is a T4 RNA ligase (Rnl2), a Chlorella virus ligase, a single-stranded DNA ligase, or a T4 DNA ligase. In some instances, generating the ligation products comprises ligating the first probe to the second probe by chemical ligation. In some instances, the releasing the ligation products from the target nucleic acids in (f) occurs during the releasing the one or more blocking probes from the ligation products in (g). In some instances, the releasing the ligation products from the target nucleic acids in (f) occurs before or after the releasing the one or more blocking probes from the ligation products in (g). In some instances, the releasing the ligation products comprises contacting the ligation products with a nuclease. In some instances, the nuclease comprises an RNase, optionally wherein the RNase is RNase A, RNase C, RNase H, or RNase I. In some instances, the releasing the ligation products from the target nucleic acids comprises using a reagent medium comprising a permeabilization agent, optionally wherein the permeabilization agent comprises a protease. In some instances, the protease comprises trypsin, pepsin, elastase, or Proteinase K. In some instances, the reagent medium further comprises a detergent. In some instances, the reagent medium further comprises polyethylene glycol (PEG).

In some instances, the hybridized ligation products comprise the capture probe capture domain and a sequence complementary to the genetic variant sequence. In some instances, the target nucleic acid comprises the genetic variant sequence. In some instances, the hybridized ligation products comprise the capture probe capture domain and a sequence complementary to the wild-type sequence. In some instances, the target nucleic acid comprises the wild-type sequence. In some instances, non-specific binding of first probes and second probes to the target nucleic acid in the biological sample is decreased compared to methods that do not include contacting with the endonuclease.

In some instances, the methods also include extending 3′ ends of the plurality of capture probes using the hybridized ligation products as a template, thereby generating extended capture probes. In some instances, the methods include amplifying the extended capture probes.

In some instances, the methods include determining sequences of: (i) all or a part of the hybridized ligation products or a complement thereof, and (ii) the spatial barcodes or a complement thereof, and using the determined sequences of (i) and (ii) to identify a spatial location of the target nucleic acid in the biological sample. In some instances, determining the sequences comprises sequencing. In some instances, the determining the sequences further comprises hybridizing detectable probes to the ligation products and detecting the detectable probes. In some instances, the detectable probe comprises a fluorescent label or a chromogenic label. In some instances, the methods also include determining an abundance of the target nucleic acid in the biological sample.

In some instances, the plurality of first probes and the plurality of second probes hybridize to adjacent sequences on the target nucleic acids. In some instances, the plurality of first probes and the plurality of second probes hybridize to non-adjacent sequences that are at least 1, 2, 3, 4, 5, 6, 7, 8, 9, 10, or more nucleotides away from one another on the target nucleic acids.

In some instances, the methods include generating extended first probes, wherein the extended first probes comprise a sequence substantially complementary to a sequence between the sequence hybridized to the first probes and the sequence hybridized to the second probes. In some instances, the methods include generating extended second probes using a polymerase, wherein the extended second probes comprise a sequence substantially complementary to a sequence between the sequence hybridized to the first probes and the sequence hybridized to the second probes. In some instances, each probe of the plurality of first probes further comprises a functional sequence, wherein the functional sequence is a primer sequence. In some instances, each probe of the plurality of second probes comprises the capture probe capture domain that is complementary to the capture domain. In some instances, each capture probe further comprises one or more functional domains, a unique molecular identifier (UMI), a cleavage domain, or a combination thereof.

In some instances, the capture domain comprises a homopolymeric sequence. In some instances, the capture probe comprises a poly(T) sequence. In some instances, the target nucleic acids are RNA. In some instances, the RNA is an mRNA. In some instances, the target nucleic acids are DNA. In some instances, the DNA is genomic DNA.

In some instances, the first substrate comprises the array. In some instances, the array is on a second substrate.

In some instances, the methods include aligning the first substrate with the second substrate such that at least a portion of the biological sample is aligned with at least a portion of the plurality of capture probes.

In some instances, the biological sample is a tissue sample. In some instances, the tissue sample is a solid tissue sample. In some instances, the solid tissue sample is a tissue section. In some instances, the biological sample is a fixed tissue sample. In some instances, the fixed tissue sample is a formalin-fixed paraffin-embedded (FFPE) tissue sample. In some instances, the FFPE tissue is deparaffinized and decrosslinked prior to contacting the first probe and the second probe with the FFPE tissue. In some instances, the tissue sample is a fresh frozen tissue sample. In some instances, the tissue sample is fixed and stained prior to contacting the plurality of first probes and the plurality of second probes.

In some instances, the methods include hybridizing RNA molecules from the biological sample to a plurality of second capture probes affixed to the array, each second capture probe comprising a second spatial barcode and a second capture domain. In some instances, the methods include extending 3′ ends of the plurality of second capture probes using the RNA molecules as a template. In some instances, the methods include determining sequences of: (iii) the second spatial barcodes or a complement thereof, and (iv) all or a portion of a sequence of the RNA molecules or a complement thereof, and using the determined sequences of (iii) and (iv) to determine an abundance and/or a spatial location of the RNA molecules in the biological sample.

In some instances, each probe of the plurality of second capture probes further comprises one or more second functional domains, a second unique molecular identifier (UMI), a second cleavage domain, or a combination thereof. In some instances, the second capture domain comprises a second homopolymeric sequence. In some instances, the second capture probe comprises a second poly(T) sequence. In some instances, the capture probe and the second capture probe are identical. In some instances, the spatial barcode identifies the spatial location of each capture probe of the plurality of capture probes on the array.

In another aspect, disclosed herein is a system for analyzing a target nucleic acid in a biological sample, the system comprising: (a) an array comprising a plurality of capture probes, wherein a capture probe of the plurality of capture probes comprises: (i) a spatial barcode and (ii) a capture domain; (b) a first probe and a second probe, wherein the first probe and the second probe each comprises a sequence that is substantially complementary to sequences of the target nucleic acid, wherein the second probe comprises a capture probe capture domain that is complementary to the capture domain, and wherein the first probe and the second probe are configured to be ligated together to form a ligation product upon hybridization to the target nucleic acid; and (c) an endonuclease that is configured to cleave a mismatched first probe and/or a mismatched second probe that is hybridized to the target nucleic acid.

In some instances, the first probe and the second probe each comprises a sequence that is substantially complementary to adjacent sequences of the target nucleic acid and wherein the first probe and the second probe are capable of being ligated together to form a ligation product upon hybridization to the target nucleic acid. In some instances, the first probe and the second probe each comprises a sequence that is substantially complementary to non-adjacent sequences of the target nucleic acid. In some instances, the array and the biological sample are on a first substrate. In some instances, the biological sample is on a first substrate and the array is on a second substrate. In some instances, the system also includes a support device configured to retain the first substrate and the second substrate. In some instances, the system also includes an alignment mechanism on the support device to align the first substrate and the second substrate. In some instances, the system also includes a reagent medium for permeabilizing the biological sample.

In another aspect, disclosed herein is a kit for analyzing a target nucleic acid in a biological sample, the kit comprising: (a) an array comprising a plurality of capture probes, wherein a capture probe of the plurality of capture probes comprises: (i) a spatial barcode and (ii) a capture domain; (b) a first probe and a second probe, wherein the first probe and the second probe each comprises a sequence that is substantially complementary to sequences of the target nucleic acid, wherein the second probe comprises a capture probe capture domain that is complementary to the capture domain; (c) an endonuclease that is configured to cleave a mismatched first probe and/or a mismatched second probe that is hybridized to the target nucleic acid; and (d) instructions for performing any one of the methods provided herein.

In some instances, the first probe and the second probe each comprises a sequence that is substantially complementary to adjacent sequences of the target nucleic acid and wherein the first probe and the second probe are capable of being ligated together to form a ligation product upon hybridization to the target nucleic acid. In some instances, the first probe and the second probe each comprises a sequence that is substantially complementary to non-adjacent sequences of the target nucleic acid, and the kit further comprises a polymerase and a plurality of dNTPs. In some instances, the array and the biological sample are on a first substrate. In some instances, the biological sample is on a first substrate and the array is on a second substrate. In some instances, the kit also includes a support device configured to retain the first substrate and the second substrate. In some instances, the kit also includes an alignment mechanism on the support device to align the first substrate and the second substrate. In some instances, the kit also includes a reagent medium for permeabilizing the biological sample.

In another aspect, disclosed herein is a method of detecting a target nucleic acid in a biological sample, the method comprising: (a) separating the biological sample into a plurality of partitions, wherein the plurality of partitions comprises a plurality of gel beads, wherein a partition of the plurality of partitions comprises a gel bead of the plurality of gel beads, wherein the gel bead comprises a capture probe comprising a cell barcode and a capture domain; (b) hybridizing a plurality of padlock probes to a plurality of target nucleic acids of the biological sample, thereby generating hybridized padlock probes, wherein the biological sample comprises a plurality of target nucleic acids, wherein each probe of the plurality of padlock probes comprises a sequence complementary to a target nucleic acid of the plurality of target nucleic acids, each target nucleic acid comprising either a wild-type sequence or a genetic variant sequence, wherein the plurality of padlock probes is configured to hybridize to both the wild-type sequence and the genetic variant sequence; (c) hybridizing a plurality of blocking oligonucleotides to the hybridized padlock probes, wherein the plurality of blocking oligonucleotides hybridizes to a sequence of the hybridized padlock probes that is not hybridized to the target nucleic acids, thereby generating protected padlock probes; (d) contacting an endonuclease with the protected padlock probes, wherein the endonuclease cleaves protected padlock probes that are hybridized to target nucleic acids and include a mismatch; and (c) generating circularized padlock probes.

In some instances, each probe of the plurality of padlock probes comprises a sequence complementary to the genetic variant sequence. In some instances, each probe of the plurality of padlock probes comprises one of four different nucleotide sequences within the region configured to hybridize to the target nucleic acid. In some instances, each probe of the plurality of padlock probes comprises identical sequences. In some instances, the endonuclease generates a nick in the protected padlock probes. In some instances, the endonuclease cleaves the protected padlock probes that are hybridized to the genetic variant sequence that includes the mismatch. In some instances, the endonuclease cleaves the protected padlock probes that are hybridized to the wild-type sequence that includes the mismatch. In some instances, the endonuclease is an S1 nuclease.

In some instances, the regions of the hybridized padlock probes that are not hybridized to the target nucleic acid are single-stranded regions. In some instances, the plurality of blocking oligonucleotides comprises one or more sequences complementary to the single-stranded regions of the hybridized padlock probes. In some instances, the methods also include releasing the plurality of blocking oligonucleotides. In some instances, releasing the plurality of blocking oligonucleotides comprises contacting the plurality of blocking oligonucleotides with a restriction endonuclease or an endoribonuclease. In some instances, releasing the plurality of blocking oligonucleotides comprises heating the plurality of blocking oligonucleotides. In some instances, releasing the plurality of blocking oligonucleotides comprises contacting the plurality of blocking oligonucleotides with a uracil-DNA glycosylase and a second endonuclease. In some instances, the second endonuclease comprises Endonuclease VIII, Endonuclease III, Endonuclease V, UNG1, UNG2, SMUG1, thymine-DNA glycosylase (TDG), or methyl CpG binding domain-4 (MBD4).

In some instances, circularizing the padlock probes comprises ligating the padlock probes by enzymatic ligation, wherein the enzymatic ligation is performed using a ligase. In some instances, the ligase is a T4 RNA ligase (Rnl2), a Chlorella virus ligase, a single-stranded DNA ligase, or a T4 DNA ligase. In some instances, circularizing the padlock probes comprises ligating the padlock probes by chemical ligation.

In some instances, the methods also include releasing the circularized padlock probes from the target nucleic acids. In some instances, releasing the circularized padlock probes from the target nucleic acids occurs before or after releasing the plurality of blocking oligonucleotides from the protected padlock probes. In some instances, releasing the circularized padlock probes from the target nucleic acids occurs during releasing the plurality of blocking oligonucleotides from the protected padlock probes. In some instances, releasing the circularized padlock probes from the target nucleic acids comprises contacting the circularized padlock probes with a nuclease. In some instances, the nuclease comprises an RNase, optionally wherein the RNase is RNase A, RNase C, RNase H, or RNase I. In some instances, the circularized padlock probes comprise a sequence complementary to the genetic variant sequence.

In some instances, the target nucleic acid comprises the genetic variant sequence. In some instances, the circularized padlock probes comprise a sequence complementary to the wild-type sequence. In some instances, the target nucleic acid comprises the wild-type sequence.

In some instances, non-specific binding of padlock probes to the target nucleic acid in the biological sample is decreased compared to methods that do not include contacting with the endonuclease.

In some instances, the methods also include amplifying the circularized padlock probes. In some instances, the amplifying is by rolling circle amplification.

In some instances, the methods include determining the sequences of: (i) all or part of the padlock probes or a complement thereof, and (ii) the cell barcodes or a complement thereof, and using the determined sequences of (i) and (ii) to identify a spatial location of the target nucleic acid in the biological sample. In some instances, the determining the sequences comprises sequencing.

In some instances, the methods also include hybridizing detectable probes to the circularized padlock probes and detecting the detectable probes to identify a spatial location of the target nucleic acid in the biological sample. In some instances, the detectable probe comprises a fluorescent label or a chromogenic label. In some instances, the methods include determining an abundance of the target nucleic acid in the biological sample.

In some instances, the target nucleic acids are RNA. In some instances, the RNA is an mRNA. In some instances, the target nucleic acids are DNA. In some instances, the DNA is genomic DNA.

In some instances, the biological sample is a tissue sample. In some instances, the tissue sample is a solid tissue sample. In some instances, the solid tissue sample is a tissue section. In some instances, the biological sample is a fixed tissue sample. In some instances, the fixed tissue sample is a formalin-fixed paraffin-embedded (FFPE) tissue sample. In some instances, the FFPE tissue is deparaffinized and decrosslinked prior to the hybridizing the plurality of padlock probes to the plurality of target nucleic acids of the FFPE tissue. In some instances, the tissue sample is a fresh frozen tissue sample. In some instances, the tissue sample is fixed and stained prior to contacting the plurality of first probes and the plurality of second probes. In some instances, the genetic variant sequence is a single nucleotide polymorphism.

In another aspect, disclosed herein is a method for determining a location of a genetic variant (e.g., a single nucleotide polymorphism (SNP) or mutation) in a nucleic acid in a biological sample, the method comprising: (a) contacting a first probe and a second probe with the biological sample on a first substrate, wherein the first probe and/or the second probe each comprise one or more sequences complementary to the SNP, and wherein the second probe comprises a capture probe capture domain; (b) hybridizing the first probe and the second probe to the nucleic acid; (c) protecting a 5′ free end of the first probe and a 3′ free end of the second probe; (d) adding an endonuclease to the biological sample, wherein the endonuclease cleaves one or more probes that hybridizes to the nucleic acid, wherein the one or more probes is not complementary to the SNP; (c) generating a ligation product by ligating the first probe and the second probe; (f) releasing the ligation product from the nucleic acid; (g) hybridizing the ligation product to a capture domain of a capture probe affixed to an array comprising a plurality of capture probes, wherein a capture probe comprises: (i) a spatial barcode and (ii) a capture domain; and (h) determining sequences of (i) all or a part of the ligation product hybridized to the capture domain, or a complement thereof, and (ii) the spatial barcode, or a complement thereof, and using the determined sequence of (i) and (ii) to identify the location of the SNP in the nucleic acid in the biological sample.

In some instances, the methods include determining the abundance of the SNP in the biological sample.

In another aspect, disclosed herein is a method for decreasing non-specific binding of a ligation product to a capture domain, the method comprising: (a) contacting a first probe and a second probe with the biological sample on a first substrate, wherein the first probe and/or the second probe each comprise one or more sequences complementary to the SNP, and wherein the second probe comprises a capture probe capture domain; (b) hybridizing the first probe and the second probe to the nucleic acid; (c) protecting a 5′ free end of the first probe and a 3′ free end of the second probe; (d) adding an endonuclease to the biological sample, wherein the endonuclease cleaves one or more probes that hybridizes to the nucleic acid, wherein the one or more probes is not complementary to the SNP; (c) generating a ligation product by ligating the first probe and the second probe; (f) releasing the ligation product from the nucleic acid; and (g) hybridizing the ligation product to a capture domain of a capture probe affixed to an array comprising a plurality of capture probes, wherein a capture probe comprises: (i) a spatial barcode and (ii) a capture domain, thereby decreasing non-specific binding of a ligation product to a capture domain.

In some instances, the non-specific binding of a ligation product to a capture domain is decreased compared to methods that do not include the endonuclease. In some instances, the endonuclease generates a nick in the first mismatched probe and/or the second mismatched probe. In some instances, the endonuclease is an S1 endonuclease. In some instances, step (c) occurs before step (d).

In some instances, the 5′ free end of the first probe and/or the 3′ end of the second probe is hybridized to a blocking probe. In some instances, the blocking probe comprises a poly(T) sequence. In some instances, the blocking probe further comprises a hairpin sequence located 5′ of the blocking probe. In some instances, the blocking probe and the 5′ free end of the first probe are on one contiguous nucleic acid sequence. In some instances, the blocking probe and the 3′ free end of the second probe are on one contiguous nucleic acid sequence. In some instances, the hairpin sequence comprises a cleavable linker selected from the group consisting of a photocleavable linker, UV-cleavable linker, or an enzyme-cleavable linker. In some instances, the hairpin sequence comprises a target sequence for a restriction endonuclease.

In some instances, releasing the blocking probe from the capture probe capture domain comprises contacting the capture probe capture domain with a restriction endonuclease or an endoribonuclease. In some instances, releasing the blocking probe from the capture probe capture domain comprises increasing the temperature of the biological sample. In some instances, releasing the blocking probe occurs substantially at the same time as releasing the ligation product from the nucleic acid. In some instances, the releasing the blocking probe from the capture probe capture domain comprises contacting the hairpin sequence and/or the blocking probe with a uracil-DNA glycosylase (UNG) and an endonuclease. In some instances, the endonuclease is one or more of endonuclease VIII, endonuclease III, endonuclease V, UNG1/UNG2, SMUG1, thymine-DNA glycosylase (TDG), and methyl CpG binding domain-4 (MBD4). In some instances, the releasing the ligation product comprises contacting the biological sample with a nuclease. In some instances, the nuclease comprises an RNase, optionally wherein the RNase is selected from RNase A, RNase C, RNase H, or RNase I.

In some instances, the releasing the ligation product utilizes a reagent medium comprising a permeabilization agent, optionally wherein the permeabilization agent comprises a protease. In some instances, the protease is selected from trypsin, pepsin, elastase, or Proteinase K. In some instances, the reagent medium further comprises a detergent. In some instances, the reagent medium further comprises polyethylene glycol (PEG).

In some instances, the 5′ free end of the first probe and/or the 3′ free end of the second probe comprises a plurality of caged nucleotides, wherein a caged nucleotide of the plurality of caged nucleotides comprises a caged moiety that blocks the capture probe capture domain from hybridizing to the capture domain of the capture probe on the substrate. In some instances, the methods also include releasing the caged moiety from the capture probe capture domain comprises activating the caged moiety, thereby allowing the capture probe capture domain to bind to the capture domain of the capture probe on the substrate. In some instances, the activating comprises photolysis of the caged moiety. In some instances, the activating comprises exposing the caged moiety to light pulses, wherein the light is at a wavelength of about or less than 360 nm. In some instances, the caged nucleotide comprises a caged moiety selected from the group consisting of 6-nitropiperonyloxymethy (NPOM), 1-(ortho-nitrophenyl)-ethyl (NPE), 2-(ortho-nitrophenyl) propyl (NPP), diethylaminocoumarin (DEACM), and nitrodibenzofuran (NDBF). In some instances, the caged nucleotide comprises a non-naturally-occurring nucleotide selected from the group consisting of 6-nitropiperonyloxymethy (NPOM)-caged guanosine, 6-nitropiperonyloxymethy (NPOM)-caged uridine, and 6-nitropiperonyloxymethy (NPOM)-caged thymidine.

In some instances, generating the ligation product comprises ligating the first probe to the second probe using enzymatic ligation, wherein the enzymatic ligation utilizes a ligase. In some instances, the ligase is one or more of a T4 RNA ligase (Rnl2), a Chlorella virus ligase, a single-stranded DNA ligase, or a T4 DNA ligase. In some instances, generating the ligation product comprises ligating the first probe to the second probe using chemical ligation. In some instances, the methods also include extending a 3′ end of the capture probe using the ligation product as a template, generating an extended capture probe. In some instances, the methods also include amplifying the extended capture probe.

In some instances, the determining step comprises sequencing. In some instances, the determining step comprises: hybridizing a detectable probe to the ligation product; and detecting the detectable probe. In some instances, the detectable probe comprises a fluorescent label or a chromogenic label.

In some instances, the contacting the first probe and the second probe with the biological sample comprises adding at least 5000 or more probe pairs, wherein a probe pair of the at least 5000 or more probe pairs comprises the first probe, the second probe, the mismatched first probe, and/or the mismatched second probe. In some instances, the contacting the first probe and the second probe with the biological sample comprises adding at least 100 or more probe pairs, wherein a probe pair of the at least 100 or more probe pairs comprises the first probe, the second probe, the mismatched first probe, and/or the mismatched second probe.

In some instances, the first probe and the second probe hybridize to adjacent sequences on the nucleic acid. In some instances, the first probe and the second probe hybridize to non-adjacent sequences that are at least 1, 2, 3, 4, 5, 6, 7, 8, 9, 10, or more nucleotides away from one another. In some instances, the methods also include generating an extended first probe, wherein the extended first probe comprises a sequence substantially complementary to a sequence between the sequence hybridized to the first probe and the sequence hybridized to the second probe. In some instances, the methods also include generating an extended second probe using a polymerase, wherein the extended second probe comprises a sequence substantially complementary to a sequence between the sequence hybridized to the first probe and the sequence hybridized to the second probe.

In some instances, the first probe further comprises a functional sequence, wherein the functional sequence is a primer sequence. In some instances, the capture probe further comprises one or more functional domains, a unique molecular identifier (UMI), a cleavage domain, or combinations thereof. In some instances, the capture domain comprises a homopolymeric sequence. In some instances, the capture probe comprises a poly(T) sequence.

In some instances, the nucleic acid is RNA. In some instances, the RNA is an mRNA. In some instances, the nucleic acid is DNA. In some instances, the DNA is genomic DNA.

In some instances, the first substrate comprises the array. In some instances, the array is on a second substrate. In some instances, the methods include aligning the first substrate with the second substrate such that at least a portion of the biological sample is aligned with at least a portion of the plurality of capture probes.

In some instances, the biological sample is a tissue sample. In some instances, the tissue sample is a solid tissue sample. In some instances, the solid tissue sample is a tissue section. In some instances, the biological sample is a fixed tissue sample. In some instances, the fixed tissue sample is a formalin-fixed paraffin-embedded (FFPE) tissue sample. In some instances, the FFPE tissue is deparaffinized and decrosslinked prior to step (a). In some instances, the tissue sample is a fresh frozen tissue sample. In some instances, the tissue sample is fixed and stained prior to step (a).

In some instances, the methods also include affixing an RNA molecule to a second capture probe comprising a second spatial barcode and second capture domain. In some instances, the affixing comprises hybridizing the RNA molecule to the second capture domain. In some instances, the methods include extending a 3′ end of the second capture probe using the RNA molecule as a template. In some instances, the methods also include determining (iii) a sequence of the second spatial barcode or a complement thereof, and (iv) all or a portion of a sequence of the RNA molecule, or a complement thereof, and using the determined sequences of (iii) and (iv) to determine abundance and/or location of the RNA molecule in the biological sample.

In some instances, the second capture probe further comprises one or more second functional domains, a second unique molecular identifier (UMI), a second cleavage domain, or combinations thereof. In some instances, the second capture domain comprises a second homopolymeric sequence. In some instances, the second capture probe comprises a second poly(T) sequence. In some instances, the capture probe and the second capture probe are identical.

In another aspect, disclosed herein is a system for analyzing a nucleic acid in a biological sample, the system comprising: (a) an array comprising a plurality of capture probes, wherein a capture probe of the plurality of capture probes comprises: (i) a spatial barcode and (ii) a capture domain; (b) a first probe and a second probe, wherein the first probe and the second probe each comprise a sequence that is substantially complementary to adjacent sequences of the nucleic acid, wherein the second probe comprises a capture probe binding domain, and wherein the first probe and the second probe are capable of being ligated together to form a ligation product; and (c) an endonuclease that cleaves a mismatched first probe and/or a mismatched second probe when hybridized to the nucleic acid, wherein the mismatched first probe differs from the first probe at the one or more sequences that are substantially complementary to sequences of the nucleic acid by at least one nucleotide.

In some instances, the array is on a first substrate. In some instances, the array is on a second substrate. In some instances, the system includes a support device configured to retain a first substrate and the second substrate, wherein the biological sample is placed on the first substrate. In some instances, the system also includes a second reagent medium for permeabilizing the biological sample. In some instances, the system further includes an alignment mechanism on the support device to align the first substrate and the second substrate.

In another aspect, disclosed herein is a kit for analyzing a nucleic acid in a biological sample, the kit comprising: (a) an array comprising a plurality of capture probes, wherein a capture probe of the plurality of capture probes comprises: (i) a spatial barcode and (ii) a capture domain; (b) a first probe and a second probe, wherein the first probe and the second probe each comprise a sequence that is substantially complementary to adjacent sequences of the nucleic acid, wherein the second probe comprises a capture probe binding domain, and wherein the first probe and the second probe are capable of being ligated together to form a ligation product; and (c) an endonuclease that cleaves a mismatched first probe and/or a mismatched second probe when hybridized to the nucleic acid, wherein the mismatched first probe differs from the first probe at the one or more sequences that are substantially complementary to sequences of the nucleic acid by at least one nucleotide, and (d) instructions for performing any of the methods disclosed herein.

In some instances, the array is on a first substrate. In some instances, the array is on a second substrate. In some instances, the kit includes a support device configured to retain a first substrate and the second substrate, wherein the biological sample is placed on the first substrate. In some instances, the kit includes a second reagent medium for permeabilizing the biological sample. In some instances, the kit includes an alignment mechanism on the support device to align the first substrate and the second substrate.

In another aspect, disclosed herein is a method of detecting a nucleic acid in a biological sample, the method comprising: (a) separating the biological sample into a plurality partitions, wherein a partition of the plurality of partitions comprises a plurality of gel beads, wherein a gel bead of the plurality of gel beads comprises a capture probe comprising a cell barcode and a capture domain; (b) hybridizing a padlock probe to the nucleic acid; (c) hybridizing an oligonucleotide that hybridizes to the padlock probe outside of the sequences hybridized to the nucleic acid; (d) adding an endonuclease to the partition, wherein the endonuclease cleaves a mismatched nucleotide of the padlock probe when the padlock probe is hybridized to the nucleic acid; (e) generating a circularized padlock probe; and (f) determining (i) all or part of the sequence of the padlock probe, or a complement thereof, and (ii) the sequence of the cell barcode, or a complement thereof, and using the determined sequences of (i) and (ii) to detect the nucleic acid in the biological sample.

In some instances, the nucleic acid comprises a SNP. In some instances, the endonuclease generates a nick in the first mismatched probe and/or the second mismatched probe. In some instances, the endonuclease is an S1 endonuclease. In some instances, generating the circularized padlock probe comprises ligating the padlock probe. In some instances, the methods include amplifying the circularized padlock probe. In some instances, the methods also include amplifying comprises rolling circle amplification. In some instances, the determining step comprises sequencing. In some instances, the determining step comprises: hybridizing a detectable probe to the ligation product; and detecting the detectable probe. In some instances, the detectable probe comprises a fluorescent label or a chromogenic label.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a sequence is at least 50%, 55%, 60%, 65%, 70%, 75%, 80%, 85%, 90%, 95%, 97%, 98%, or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, and/or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection, but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows an exemplary workflow for performing templated capture and producing a ligation product.

FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe and an analyte capture agent.

FIG. 12 shows a schematic of a first probe and a second probe affixed to a nucleic acid target having a SNP.

FIGS. 13A-13F show interaction of a mismatched probe pair (FIGS. 13A-13C) or a complementary probe pair (FIGS. 13D-13F) with an endonuclease.

FIG. 14 shows a schematic of digestion of the single-stranded handles of ligation probes.

FIG. 15 shows a schematic of protecting digestion of the single-stranded non-target complementary handles of ligation probes using complementary oligonucleotides.

FIG. 16 shows exemplary workflows and interaction of a mismatched probe pair or a complementary probe pair with an endonuclease described herein.

FIG. 17 shows an exemplary workflow including a padlock probe described herein.

### DETAILED DESCRIPTION

### A. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid)) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Patent Application Publication Nos. WO2018/091676, WO2020/176788, WO2017/144338, and WO2016/057552; Non-patent literature references Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I)(b) of PCT Patent Application Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Patent Application Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaeon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed, e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin-fixed biological sample, which can be optionally dehydrated via sucrose gradient and/or embedded in OCT compound, is then frozen e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin-embedded. Thus, in preferred embodiments, a fixed frozen biological sample is not deparaffinized. In some embodiments, a fixed frozen biological sample is rehydrated in an ethanol gradient.

In some instances, the biological sample (e.g., a fixed frozen tissue sample) is treated with a citrate buffer. Citrate buffer can be used to decrosslink antigens and fixation medium in the biological sample for antigen retrieval. Thus, any suitable decrosslinking agent can be used in addition to or alternatively to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, the sample is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, an acid, and a soluble organic compound that preserves morphology of biomolecules. PAXgene provides a two-reagent fixative system in which tissue is firstly fixed in a solution containing methanol and acetic acid, then stabilized in a solution containing ethanol. See, e.g., Ergin B. et al., J Proteome Res. 2010 Oct 1;9(10):5188-96; Kap M. et al., PLOS One.; 6(11):e27704 (2011); and Mathieson W. et al., Am J Clin Pathol.; 146(1):25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample, is fixed, for example in methanol, acetone, acetone-methanol, PFA, and/or PAXgene or is formalin-fixed and paraffin-embedded (FFPE). In some embodiments, the biological sample includes intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. However, by utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Patent Application Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes as disclosed herein to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′,6-diamidino-2-phenylindole), eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Patent Application Publication No. WO2020/176788 and/or U.S. Patent Application Publication No. 2020/0277663, which is herein incorporated by reference. Briefly, in any of the methods described herein, the method includes a step of permeabilizing the biological sample. For example, the biological sample can be permeabilized to facilitate transfer of extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is herein incorporated herein by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Patent Application Publication No. WO2020/176788 and/or U.S. Patent Application Publication No. 2020/0277663, which is herein incorporated by reference. Generation of capture probes can be achieved by any appropriate method, including those described in Section (II)(d)(ii) of PCT Patent Application Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues in the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the array of capture probes is on (e.g., affixed to) a second substrate. In this configuration, one or more analytes or analyte derivatives (e.g., intermediate agents, e.g., ligation products) are then released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described, e.g., in U.S. Patent Application Publication No. 2021/0189475 and PCT Patent Application Publication Nos. WO2021/252747 A1, WO2022/061152 A2, and WO2022/140028 A1, each of which is herein incorporated by reference.

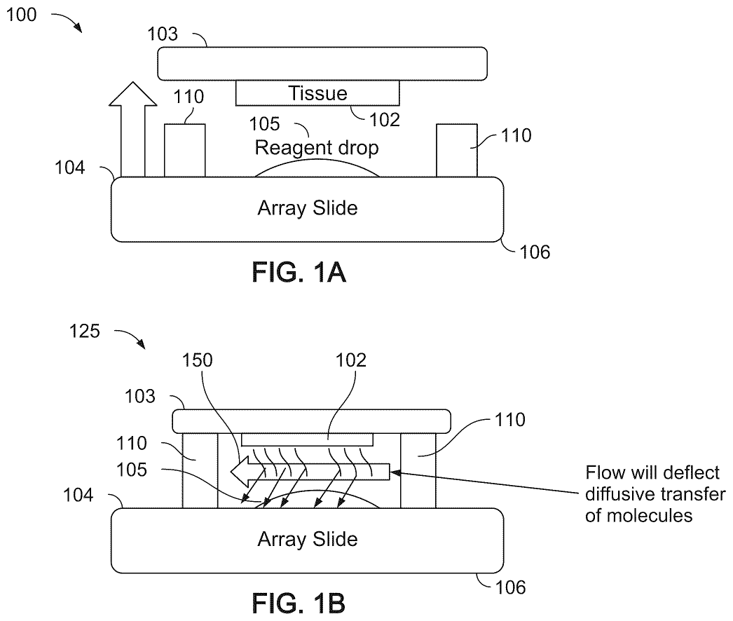

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a drop of liquid reagent (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents, e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents, e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent, e.g., ligation product) from the biological sample is performed actively (e.g., electrophoretic, by applying an electric field to promote migration). Exemplary methods of electrophoretic migration are described in WO2020/176788, and U.S. Patent Application Publication No. 2021/0189475, each of which is hereby incorporated by reference.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns (μm) and about 1 millimeters (mm), e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm, measured in a direction orthogonal to the surface of the first substrate that supports the biological sample and the surface of the second substrate including the capture probes. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents, e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents, e.g., ligation products) for spatial analysis. A partially or fully sealed chamber 150 resulting from the one or more spacers 110, the first substrate (e.g., slide 103), and the second substrate (e.g., slide 104) may reduce or prevent flow from undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Publication No. 2021/0189475, and PCT Patent Application Publication No. WO2022/061152 A2, each of which is incorporated by reference in its entirety.

In some embodiments, the sample holder can include a first member including a first retaining mechanism configured to retain a first substrate including a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

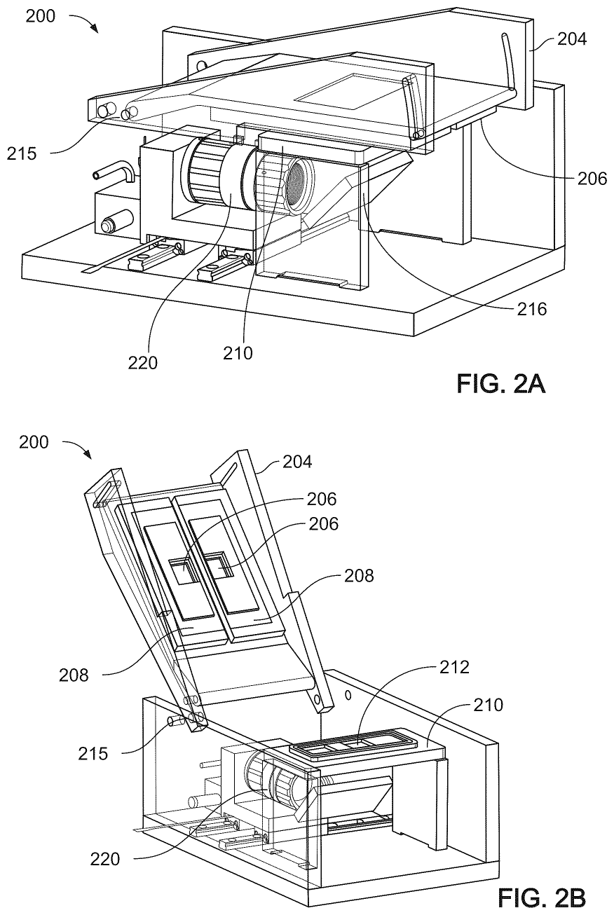

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents, e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

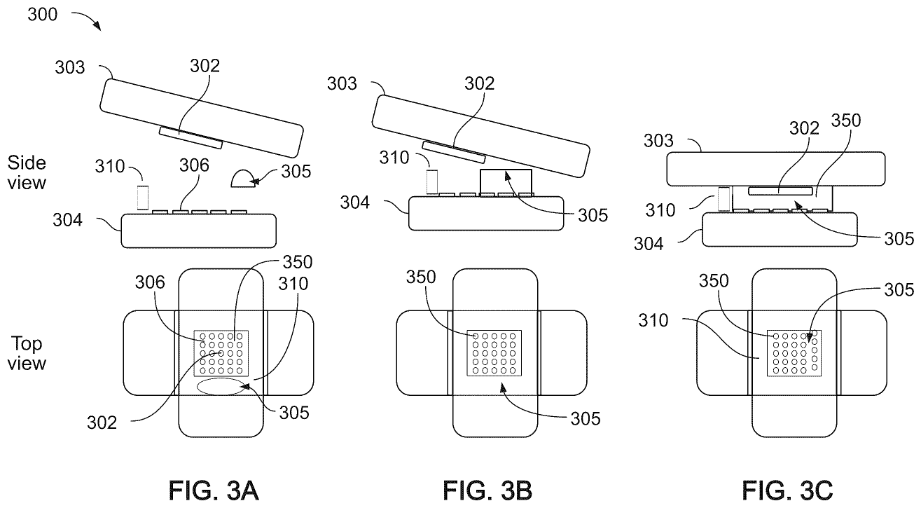

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., the slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate including the spacer 310, it should be understood that an exemplary angled closure workflow can include the second substrate angled over (superior to) the first substrate and the first substrate including the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

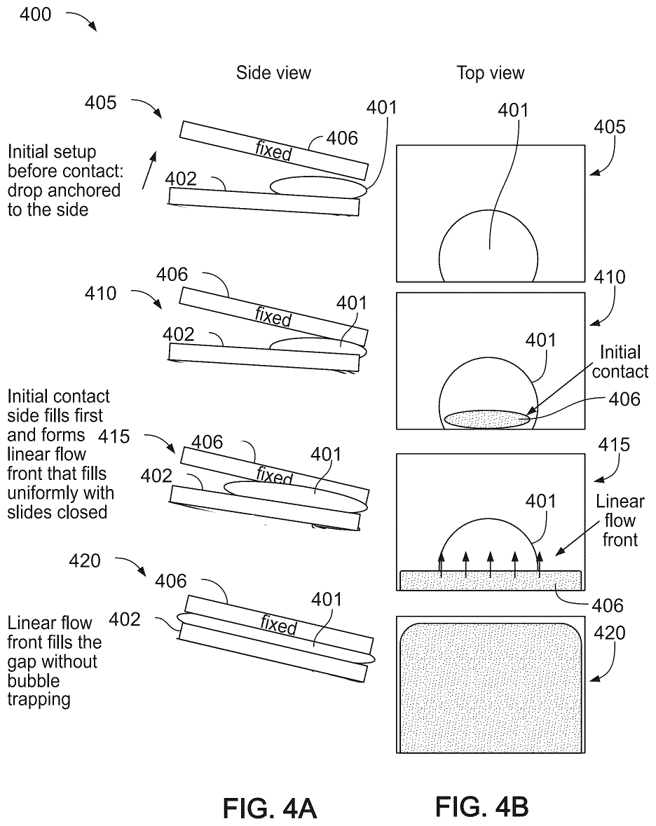

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) includes a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, or SDS), and enzymes (e.g., trypsin or other proteases (e.g., Proteinase K)). In some embodiments, the detergent is an anionic detergent (e.g., SDS or N-lauroylsarcosine sodium salt solution).

In some embodiments, the reagent medium includes a lysis reagent. Lysis solutions can include ionic surfactants such as, for example, sarkosyl, and SDS. More generally, chemical lysis agents can include, without limitation, organic solvents, chelating agents, detergents, surfactants, and chaotropic agents. In some embodiments, the reagent medium includes a protease. Exemplary proteases include, e.g., pepsin, trypsin, elastase, and Proteinase K. In some embodiments, the reagent medium includes a nuclease. In some embodiments, the nuclease includes an RNase. In some embodiments, the RNase includes RNase A, RNase C, RNase H, and/or RNase I. In some embodiments, the reagent medium includes one or more of SDS or a sodium salt thereof, Proteinase K, pepsin, N-lauroylsarcosine, and RNase.

In some embodiments, the reagent medium includes polyethylene glycol (PEG). In some embodiments, the molecular weight of the PEG is from about 2K to about 16K. In some embodiments, the molecular weight of the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b) (vii) of PCT Patent Application Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Patent Application Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the capture analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Patent Application Publication No. WO2020/176788 and/or U.S. Patent Application Publication No. 2020/0277663, which is herein incorporated by reference. Some quality control measures are described in Section (II)(h) of PCT Patent Application Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Patent Application Publication No. WO2020/176788 and/or U.S. Patent Application Publication No. 2020/0277663, which is herein incorporated by reference. Exemplary features and geometric attributes of an array can be found in Sections (II)(d)(i), (II)(d)(iii), and (II)(d)(iv) of PCT Patent Application Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) including capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Patent Application Publication No. WO2020/176788 and/or U.S. Patent Application Publication No. 2020/0277663, which is herein incorporated by reference.

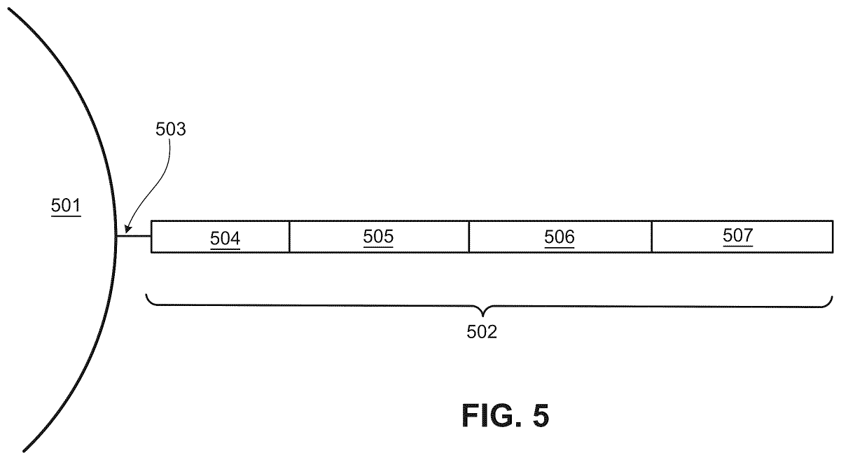

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a RI primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

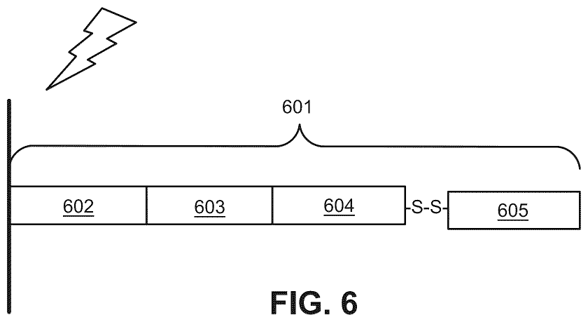

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example, a spatial barcode and a capture domain.

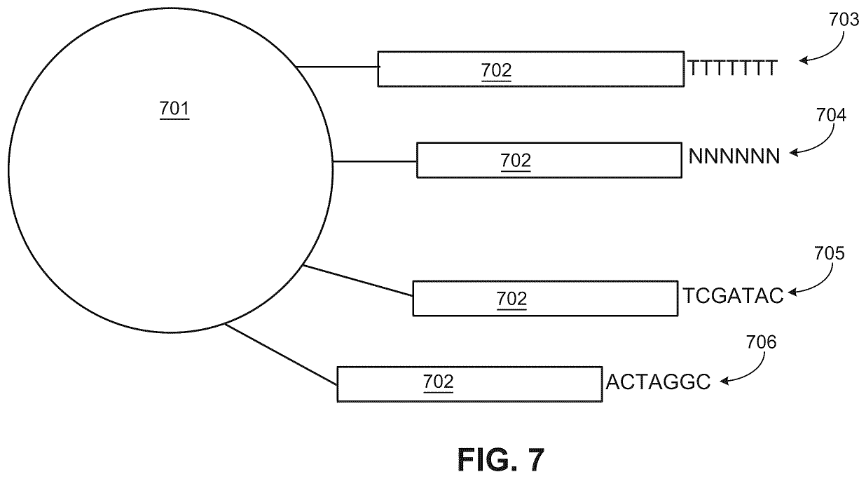

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequence 504 are common to all of the probes attached to a given feature. In some embodiments, the UMI sequence 506 of a capture probe attached to a given feature is different from the UMI sequence of a different capture probe attached to the given feature.

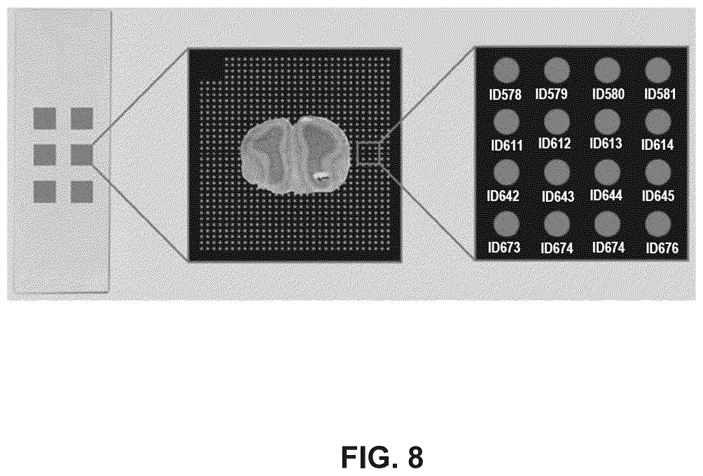

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Patent Application Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell or cell nucleus in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells or cell nuclei in a biological sample) for use in spatial analysis. In some embodiments, after attaching and/or introducing a molecule having a barcode to a biological sample, the biological sample can be physically separated (e.g., dissociated) into single cells, single cell nuclei, or cell groups for analysis. Some such methods of spatial analysis are described in Section (III) of PCT Patent Application Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug 21; 45(14):e128, which is herein incorporated by reference. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides can create a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location, and optionally, the abundance of the analyte in the biological sample.

In some instances, one or both of the oligonucleotides may hybridize to genomic DNA (gDNA) which can lead to false positive sequencing data from ligation events on gDNA (off target) in addition to the desired (on target) ligation events on target nucleic acids, (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

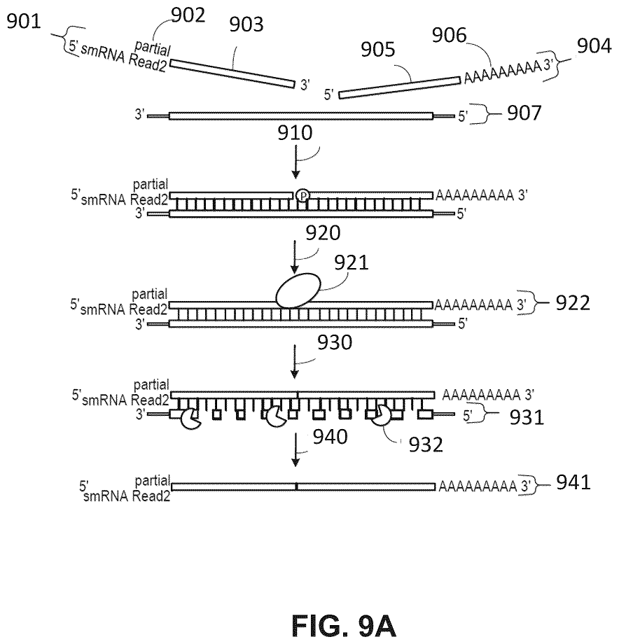

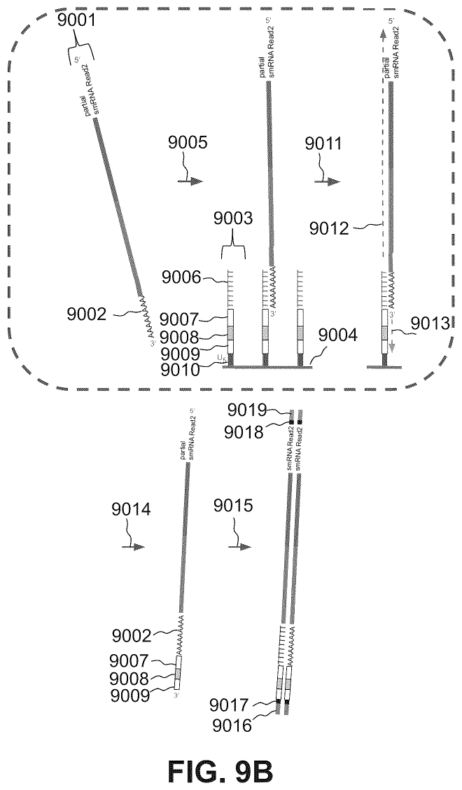

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and compositions for spatial detection using templated ligation have been described in PCT Patent Application Publication No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily bind to target analytes (i.e., compared to no permeabilization). In some embodiments, reverse transcription (RT) reagents can be added to permeabilize biological samples. Incubation with the RT reagents can be used to extend the capture probes 9011 to produce spatially-barcoded full-length cDNA 9012 and 9013 from the captured analytes (e.g., polyadenylated mRNA). Second strand reagents (e.g., second strand primers, enzymes, etc.) can be added to the biological sample to initiate second strand synthesis.

In some embodiments, methods provided herein include permeabilization of the biological sample such that the capture probe can more easily capture the ligation products (i.e., compared to no permcabilization). In some embodiments, polymerization (e.g., reverse transcription (RT)) reagents can be added to permeabilized biological samples. Incubation with the RT reagents can be used to extend the capture probes 9011 to produce spatially-barcoded full-length cDNA 9012 and 9013 from the captured ligation products (e.g., polyadenylated ligation products).

In some embodiments, the extended ligation products can be denatured 9014, released from the capture probe, and transferred (e.g., to a clean tube) for amplification, and/or library construction. The spatially-barcoded ligation products can be amplified 9015 via PCR prior to library construction. P5 9016 and P7 9019 sequences can be used for sequencing, while i5 9017 and i7 9018 sequences can be used as sample indexes. The amplicons can then be sequenced using paired-end sequencing using TruSeq Read 1 and TruSeq Read 2 as sequencing primer sites for Illumina sequencers. Other sequencing systems can be used, all that is required is the sequences specific to a particular instrument and workflow is incorporated into the sequencing libraries.

In some embodiments, in addition to the detection of genetic variants in a biological sample, detection of one or more other analytes (e.g., protein analytes) can be performed, either sequentially or concurrently, using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Patent Application Publication No. WO2020/176788 and/or Section (II)(b)(viii) U.S. Patent Application Publication No. 2020/0277663, which is herein incorporated by reference.

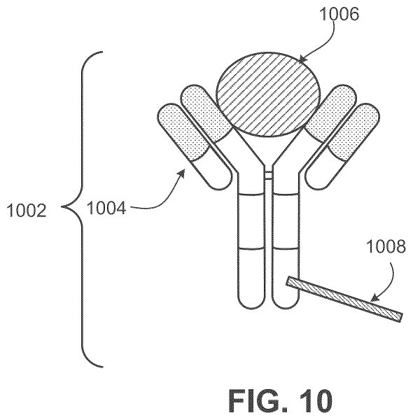

FIG. 10 is a schematic diagram of an exemplary analyte capture agent 1002 comprised of an analyte binding moiety 1004 and an analyte binding moiety barcode domain 1008. The analyte binding moiety 1004 is a molecule capable of binding to an analyte 1006 and the analyte capture agent 1002 is capable of interacting with a spatially-barcoded capture probe, e.g., on an array. The analyte binding moiety 1004 can bind to the analyte 1006 with high affinity and/or with high specificity. The analyte capture agent 1002 can include: (i) an analyte binding moiety barcode domain 1008, which serves to identify the analyte binding moiety, and (ii) a capture domain, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen binding fragment).

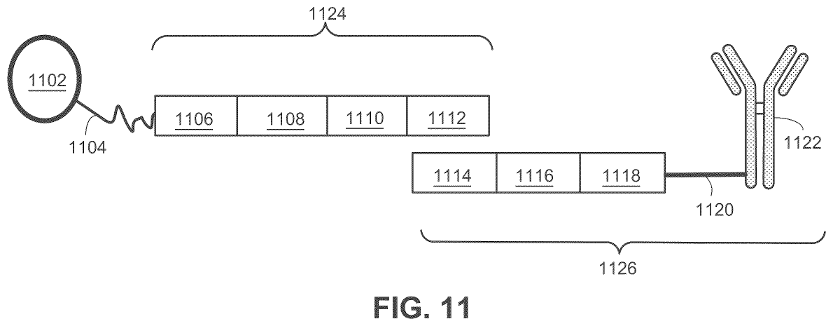

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequence 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include a functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable linker, thermal-cleavable linker, or an enzyme cleavable linker. In some instances, the cleavable linker is a disulfide linker. A disulfide linker can be cleaved by use of a reducing agent, such as dithiothreitol (DTT), beta-mercaptoethanol (BME), or tris(2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location or a fiducial marker) of the array. Accordingly, each feature location has an “address” or location in the coordinate space of the array.

Some exemplary spatial analysis workflows are described in the Exemplary Embodiments section of PCT Patent Application Publication No. WO2020/176788 and/or U.S. Patent Application Publication No. 2020/0277663, which is herein incorporated by reference. See, e.g., the Exemplary embodiment starting with “In some non-limiting examples of the workflows described herein, the sample can be immersed . . . ” of PCT Patent Application Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022); and/or the Visium Spatial Gene Expression Reagent Kits—Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II)(e)(ii) and/or (V) of PCT Patent Application Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Patent Application Publication No. WO2020/123320, which is herein incorporated by reference.

Suitable systems for performing spatial analysis can include components such as a chamber (e.g., a flow cell or a scalable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid-state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD or CMOS) used to capture images. The systems can also optionally include one or more light sources (e.g., LED-based, diode-based, or lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Patent Application Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Patent Application Publication No. WO2020/053655 and spatial analysis methods are generally described in PCT Patent Application Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Patent Application Publication Nos. WO2020/123320, WO2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, or to determine a location of a sample or array on a substrate relative to a fiducial marker) and/or for quantitative measurements of sizes and/or distances.

### B. Spatial Analysis of Genetic Variants

Generally, targeting and capturing a particular analyte in a biological sample utilizes a capture probe that hybridizes to a common transcript sequence such as a poly(A) mRNA-like tail. Methods such as templated ligation (as disclosed herein) offer an alternative to non-discriminant capture of a common transcript sequence. See, e.g., Yeakley, PLOS One, 25;12(5):e0178302 (2017), which is incorporated by reference in its entirety. Targeted RNA capture may be an attractive alternative to poly(A) mRNA capture to interrogate spatial gene expression in a sample (e.g., an FFPE tissue sample). Compared to poly(A) mRNA capture methods, targeted RNA capture methods as described herein may be less affected by RNA degradation associated with FFPE fixation compared to methods dependent on oligo-dT capture and reverse transcription of captured mRNA. Further, targeted RNA capture methods as described herein may allow for sensitive measurement of specific genes of interest that may otherwise be missed with a whole transcriptomic approach. Targeted RNA capture can be used to capture a defined set of RNA molecules of interest, at a whole transcriptome level, or a combination thereof. When combined with the spatial methods disclosed herein, the location and abundance of the RNA targets can be determined. The methods described herein advance the field of spatial transcriptomics by applying templated ligation methods to more effectively detect genetic variants, such as single nucleotide polymorphisms (SNPs) and/or mutations. The methods described herein can be used to identify and digest mismatched probes hybridized to their targets, even at just a one base pair difference. Mismatching of a probe to its target can occur, for instance, when a wild-type probe (i.e., having sequence complementarity to a wild-type target sequence) binds to a target sequence including a genetic variant.

The methods provided herein utilize probe pairs or probe sets. In some instances, the probe pairs are designed such that each probe hybridizes to a sequence in an analyte that is specific to the analyte (e.g., compared to the entire genome). That is, in some instances, a single probe pair can be specific to (and hybridize to) a single analyte, and even a single analyte having a genetic variant. The methods disclosed herein detect genetic variants and mutations in nucleic acids, including RNA (e.g., mRNA) and DNA (e.g., genomic DNA) in various sample types. Samples that can be utilized in the methods disclosed herein include tissue samples (e.g., solid tissue samples or tissue sections), fixed tissue sample (e.g., formalin-fixed, paraffin-embedded (FFPE) tissue samples), and fresh frozen tissue samples. In some instances, with respect to fixed samples, the tissue is deparaffinized and decrosslinked prior to genetic variant detection. In some instances, the tissue sample is fixed and stained prior to genetic variant detection.

In some embodiments, templated ligation probe pairs can be designed such that one probe of a given probe pair hybridizes to a specific sequence. The other, “second” probe of the given probe pair can be designed to detect a genetic variant (e.g., a SNP or mutation). Accordingly, in some instances, multiple second probes can be designed to vary such that each of the second probes binds to a specific sequence. For example, one second probe can be designed to hybridize to a wild-type sequence, and another second probe can be designed to detect a mutated sequence. Thus, in some instances, a probe set can include one first probe and two, three, or four second probes (or vice versa).

In some instances, probe pairs can target both an intended sequence and an unintended sequence. For instance, a target nucleic acid may include a genetic variant (e.g., a SNP, a mutation, or multiple SNPs/mutations), but a probe designed to hybridize to a wild-type target may also hybridize to a target having a genetic variant (e.g., SNP or mutation). In this instance, identifying genetic variants (e.g., SNPs and mutations) can be difficult because the wild-type probe also hybridizes to the sequence having the genetic variant (with a mismatch sequence). To solve this issue, the present disclosure includes use of an enzyme (e.g., an endonuclease, e.g., an S1 nuclease) that nicks single nucleotide mismatches between a probe and a target. Such an enzyme may be used to cleave a hybridized probe (or probe pair) including a mismatched sequence. The resulting methods eliminate false positive artifacts arising from probe capture of non-wild-type targets, i.e., mismatched ligated probes bound to genetic variants of targets are cleaved and do not undergo further processing to generate data, while correctly bound ligated probes bound to wild-type targets are unaffected, thereby increasing specificity of a templated ligation assay.

Using an endonuclease to digest probes that mismatch to a target offers a number of advantages. First, some endonucleases, such as S1 nuclease, can digest RNA: DNA duplexes that have mismatches. Second, endonucleases may be more active in digesting DNA compared to RNA, thereby potentially allowing RNA molecules with the genetic variant, such as a SNP or mutation, to evade digestion. Digesting mismatched probe pairs may provide a more accurate and robust understanding of the abundance of genetic variants (e.g., SNPs or mutations) in a biological sample.

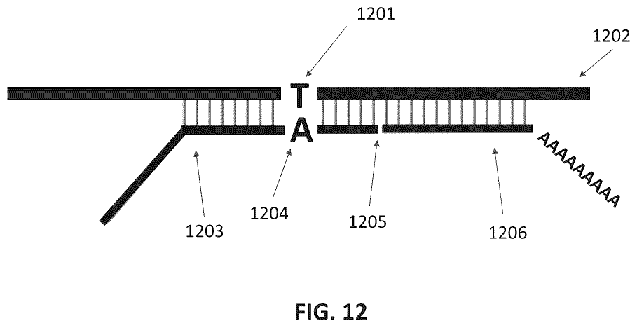

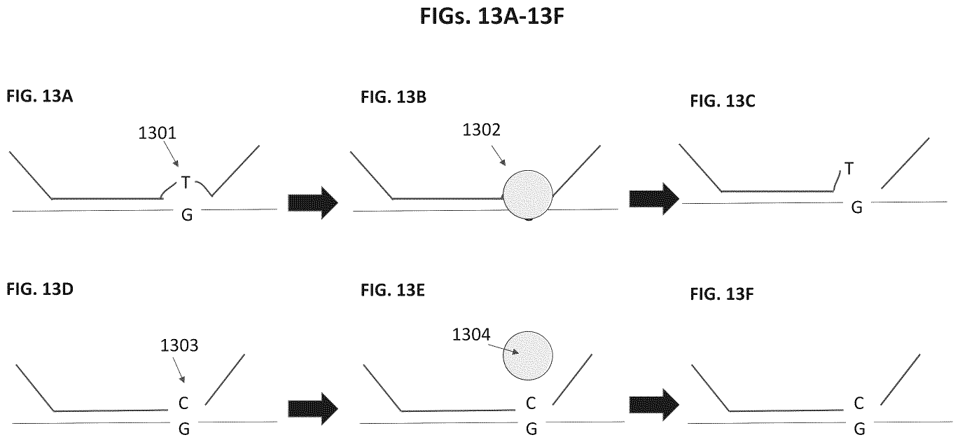

Embodiments of the present disclosure are provided in FIG. 12 and FIGS. 13A-13F. Briefly, in FIG. 12, a nucleic acid target 1202 includes a genetic variant (e.g., a SNP) 1201 depicted as a “T” nucleotide. Probes may be designed to hybridize to the nucleic acid target 1202. As shown, one probe 1203 hybridizes to the sequence including the SNP 1204, which is shown as internal to probe 1203. After the first probe 1203 and the second probe 1206 hybridize to the nucleic acid target 1202, the probes may be ligated together, e.g., by use of a ligase at nick or gap 1205.

In some instances, the first probe and/or the second probe hybridize to target sequences, but are not fully complementary to the target sequences. For instance, as shown in FIG. 13A, a probe (or probe pair) can hybridize to a sequence with one or more mismatches (shown as 1301 with a “T” mismatching with a “G”). In this instance, as shown in FIG. 13B, an endonuclease 1302 (e.g., an S1 nuclease) may be added to the biological sample, and the mismatched pairings are nicked. After a probe is nicked, as shown in FIG. 13C, the mismatched nicked probe will be excluded in downstream analysis because the nicked probe will not include either a primer from the first probe or a poly(A) tail from the second probe. Conversely, when a first probe or second probe correctly (i.e., with 100% complementary) hybridizes to a target sequence, a perfect match 1303 is made and the probe pair can then be ligated (FIG. 13D). In this instance, the S1 nuclease 1304, as shown in FIG. 13E, does not nick the intact probe, and the probe may continue to downstream analysis after generation of the ligation product, as shown in FIG. 13F.

### Probes and Blocking Oligonucleotides

Disclosed herein are nucleic acid probes and/or probe sets that are introduced into a cell or used to otherwise contact a biological sample such as a tissue sample. In some aspects, the nucleic acid probes and/or probe sets comprise at least two hybridization regions capable of hybridizing to target sequences in the target nucleic acid. In some instances, the probes are designed to target a sequence having a genetic variant such as a SNP (or more than one mutation). As used herein, the probes can be referred to as a “first probe” and a “second probe.” As will be appreciated, these terms can be interchangeable depending on the context.

In some embodiments, probe sets cover all, nearly all, or a subset of a transcriptome or a genome (e.g., a human transcriptome or a human genome). Probe sets may be designed to detect analytes in an unbiased manner. In some instances, one probe pair may be designed to target one analyte (e.g., a transcript). In some instances, more than one probe pairs (e.g., a probe pair including a first probe and a second probe) is designed to cover one analyte (e.g., transcript). For example, at least two, three, four, five, six, seven, eight, nine, ten, or more probe sets can be used to hybridize to a single analyte. Factors to consider when designing probes is presence of genetic variants (e.g., SNPs or mutations) or multiple isoforms expressed by a single gene. In some instances, the probe pair does not hybridize to the entire analyte (e.g., a transcript), but instead hybridizes to a portion of the entire analyte (e.g., a transcript). In some instances, the probe pairs comprise one first probe and multiple second probes, or vice versa.

Detection of a genetic variant can be achieved by designing the probes such that one probe includes a sequence of a wild-type sequence, and the other probe is designed to hybridize to a sequence (e.g., an adjacent sequence) that includes a genetic variant. For instance, in some embodiments, the multiple first probes are designed to include one of four different nucleotides (i.e., G, C, A, or T) at the site of the genetic variant, one for each of the multiple probes, while the second probe includes a sequence that is complementary to a wild-type sequence. In other instances, the first probe includes a sequence that is complementary to a wild-type sequence and the multiple second probes are designed to include one of four different nucleotides (i.e., G, C, A, or T) at the site of the genetic variant, one for each of the second probes. Still, in other instances, multiple nucleotide positions (e.g., 2, 3, 4, 5, 6, 7, 8, 9, 10, or more nucleotide positions) on a target analyte have a genetic variant and each may be targeted by the first probe and the second probe. In these instances, multiple probes can be designed to target the sequences, and each position of the first probe and/or second probe can be altered to target the genetic variant. That is, each position on the probe targeting the genetic variant can include an A, G, T, or C. Thus, the present disclosure has the ability to detect which genetic variant is present by designing multiple first probes and/or multiple second probes.

In some instances, about 100, about 500, about 1000, about 5000, about 10,000, about 15,000, about 20,000, or more probe pairs (e.g., a probe pair comprising: a first probe and a second probe, a first probe and multiple second probes, or multiple first probes and a second probe) are used in the methods described herein. In some instances, about 20,000 probe pairs are used in the methods described herein. In some embodiments, the subset of analytes includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700, about 800, about 900, or about 1000 analytes. In some instances, the methods disclosed herein can detect the abundance and location of at least 5,000, at least 10,000, at least 15,000, at least 20,000, or more different analytes. The analytes can be a nucleic acid. In some instances, the nucleic acid is RNA. In some instances, the RNA is an mRNA. In some instances, the nucleic acid is DNA. In some instances, the DNA is genomic DNA. Also, as further described herein, proteins can be detected as part of the one or more different analytes.

In some embodiments, the first and/or second probe includes ribonucleotides, deoxyribonucleotides, and/or synthetic nucleotides that are capable of participating in Watson-Crick type or analogous base pair interactions. In some embodiments, the first and/or second probe includes deoxyribonucleotides. In some embodiments, the first and/or second probe includes deoxyribonucleotides and ribonucleotides. In some embodiments, the first and/or second probe includes a deoxyribonucleic acid sequence that hybridizes to an analyte, and a portion of the probe is not a deoxyribonucleic acid. For example, in some embodiments, the portion of the first and/or second probe that is not a deoxyribonucleic acid is a ribonucleic acid or any other non-deoxyribonucleic acid as described herein. In some embodiments, where the first and/or second probe includes deoxyribonucleotides, hybridization of the first and/or second probe to an mRNA analyte results in a DNA:RNA hybrid. In some embodiments, the first and/or second probe includes only deoxyribonucleotides and upon hybridization of the first and/or second probe to the mRNA molecule results in a DNA:RNA hybrid.

In some embodiments, the first and/or second probe includes one or more sequences that are substantially complementary to one or more sequences of an analyte. In some embodiments, a first and/or second probe includes a sequence that is substantially complementary to a first and/or second target sequence of the analyte. In some embodiments, the sequence of the first and/or second probe that is substantially complementary to the first and/or second target sequence of the analyte is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the first and/or second target sequence in the analyte.

In some embodiments, a first and/or second probe includes a sequence that is about 10 nucleotides to about 100 nucleotides (e.g., a sequence of about 10 nucleotides to about 90 nucleotides, about 20 nucleotides to about 80 nucleotides, about 30 nucleotides to about 70 nucleotides, about 40 nucleotides to about 60 nucleotides, or about 50 nucleotides) in length. In some embodiments, a sequence of the first and/or second probe that is substantially complementary to a sequence of the analyte includes a sequence that is about 5 nucleotides to about 50 nucleotides (e.g., about 5 nucleotides to about 45 nucleotides, about 10 nucleotides to about 40 nucleotides, about 15 nucleotides to about 35 nucleotides, about 20 nucleotides to about 30 nucleotides, or about 25 nucleotides) in length.

In some embodiments, the first and/or second probe includes a functional sequence. In some embodiments, a functional sequence includes a primer sequence.

In some embodiments, the first and/or second probe includes at least two ribonucleic acid bases at the 3′ end. In some embodiments, a first or second probe includes a phosphorylated nucleotide at the 5′ end, thereby allowing for ligation between the first and second probe when hybridized to the analyte. In some embodiments, a first probe includes at least three, at least four, at least five, at least six, at least seven, at least eight, at least nine, or at least ten ribonucleic acid bases at the 3′ end.

In some embodiments, a first and/or second probe includes a capture probe capture domain. As used herein, a “capture probe capture domain” can hybridize to a capture domain of a capture probe on a spatial array. In some embodiments, a first and/or second probe includes, e.g., from a 5′ to 3′ direction, a sequence that is substantially complementary to a sequence in the analyte and a capture probe capture domain.

In some embodiments, a capture probe capture domain includes a poly(A) sequence. In some embodiments, the capture probe capture domain includes a poly-uridine sequence, a poly-thymidine sequence, or both. In some embodiments, the capture probe capture domain includes a random sequence (e.g., a random hexamer or octamer). In some embodiments, the capture probe capture domain is complementary to a capture domain of a capture probe that detects a particular target(s) or sequence of interest (a fixed or defined capture probe capture domain that is complementary to a fixed or defined capture domain of a capture probe on a spatial array).

In some instances, the methods and compositions disclosed herein include one or more oligonucleotides that are complementary to a free (i.e., non-hybridized, single-stranded) 5′ end of the first probe and/or a free 3′ end of the second probe. Because the 5′ and 3′ ends of the first and/or second probes are single-stranded, these regions may be subjected to endonuclease-mediated cleavage, thereby preventing downstream processing of the first and/or second probes. To protect the free 5′ and 3′ regions of the probes from undesired endonuclease-mediated cleavage, complementary oligonucleotides may be used to interact (e.g., hybridize) to the 5′ and 3′ regions of the probes that do not hybridize to the target nucleic acid prior to endonuclease treatment. In some instances, these oligonucleotides are referred to as blocking probes, blocking oligonucleotides, or blocking moieties. In some embodiments, a blocking probe hybridizes to the capture probe capture domain. In some embodiments, a blocking probe interacts with the functional domain (e.g., a primer). In some embodiments, a blocking probe includes a sequence that is complementary to a capture domain of a capture probe on a spatial array. In some embodiments, a blocking probe prevents digestion of the 5′ end of the first probe. In some embodiments, a blocking probe prevents digestion of the 3′ end of the second probe.

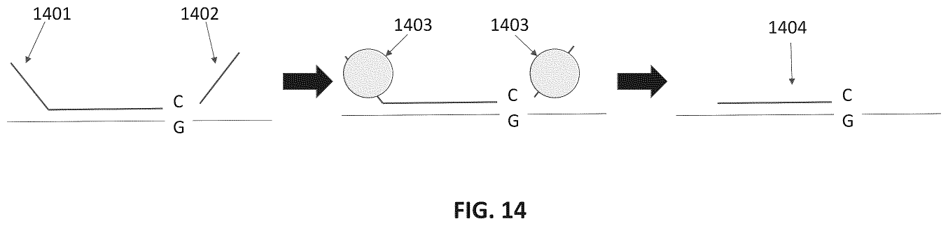

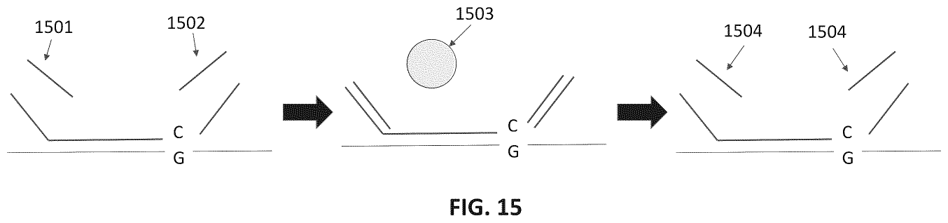

As shown in FIG. 14, in the absence of a blocking probe, S1 nuclease 1403 digests the non-target complementary single-stranded ends (e.g., a functional sequence 1401 and a poly(A) capture probe capture domain 1402) of the first and second probe. When the non-complementary single-stranded ends are digested 1404, no data can be generated because a ligation product will not include a capture probe capture domain that is used to hybridize to a capture domain of a capture probe on a spatial array. To address this potential issue, as shown in FIG. 15, blocking probes 1501 and 1502 can be used. The blocking probes may be complementary to the 5′ end of the first probe and the 3′ end of the second probe. Hybridization of the blocking probes to free ends of the first probe and the second probe generates double-stranded molecules, which are not capable of being digested by the S1 nuclease 1503. Accordingly, the free ends of the first and second probe are protected from digestion by the S1 nuclease 1503. Instead, only internal mismatches are available to be nicked. After enzyme treatment, the blocking probes can be released 1504 and washed away. In some embodiments, a blocking moiety includes a poly-uridine sequence, a poly-thymidine sequence, or both.

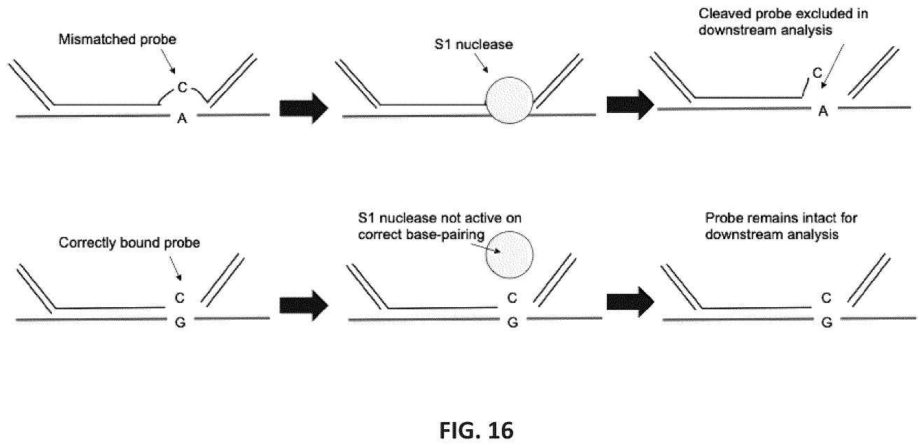

Similarly, as shown in FIG. 16, a plurality of identical first probes or a plurality of identical second probes hybridizes to target nucleic acids of a biological sample. The target nucleic acids may include either a wild-type sequence (e.g., depicted as an “A” nucleotide) or a genetic variant sequence (e.g., depicted as a “G” nucleotide). While the first or second probes are configured to hybridized to both target nucleic acids, hybridization to the wild-type sequence results in a mismatch, whereas hybridization to the genetic variant sequence results in no mismatch (i.e., correct base-pairing with 100% sequence complementarity). Endonuclease cleavage (e.g., using S1 nuclease) results in cleavage of only the mismatch, i.e., the wild-type sequence, and preservation of the probes hybridized to the genetic variant sequence. These probes that are specific to the genetic variant sequence may then proceed to downstream processing and analysis. Accordingly, the probes are specific for the detection of the genetic variant sequence. Any target nucleic acid that includes the genetic variant sequence can be detected using this assay. In the same respect, the assay can be adapted to be specific to the detection of the wild-type sequence. The probes may be configured to hybridize to the wild-type sequence with 100% complementarity and hybridize to any genetic variant with less than 100% complementarity (a mismatched sequence). Endonuclease cleavage (e.g., using S1 nuclease) may be used to cleave probes hybridized to the genetic variants, which would be excluded from downstream processing and analysis.

### Endonucleases

In some embodiments, the methods provided herein include digesting or nicking a mismatched probe hybridized to a target using an enzyme. In some embodiments, the method includes contacting the biological sample with a nuclease, such as a restriction endonuclease. In some embodiments, the sample is contacted with at least any of 1 unit (U), 2 U, 5 U, 10 U, 20 U, 30 U, 40 U, 50 U, 60 U, 70 U, 80 U, 90 U, or 100 U of a restriction endonuclease. One unit of restriction endonuclease activity is defined as the amount of enzyme (measured in units, U) that will cleave 1 μg of DNA (usually lambda DNA) to completion in 1 hour at the optimum temperature for the enzyme, usually 37° C. In some embodiments, the restriction endonuclease cleaves at a recognition sequence that is 4, 5, 6, 7, 8, or more base pairs in length.

In some instances, the enzyme (e.g., an endonuclease) generates a nick in the first mismatched probe and/or the second mismatched probe. In some instances, the endonuclease is S1 nuclease. In some embodiments, cleaving the double-stranded recognition sequence includes incubating the sample with the nuclease (e.g., a S1 nuclease). In some embodiments, the method includes incubating the biological sample with the nuclease for at least about 20 minutes, at least about 30 minutes, at least about 35 minutes, at least about 40 minutes, at least about 45 minutes, at least about 50 minutes, at least about 60 minutes, at least about 80 minutes, at least about 100 minutes, or at least about 120 minutes. In some embodiments, the incubation with the nuclease is performed for about 20-60 minutes, about 20-45 minutes, about 20-120 minutes, about 30-120 minutes, about 30-60 minutes, or about 30-90 minutes. In some embodiments, the incubation with the restriction endonuclease is performed at about 30-40° C., e.g., at 37° C.

In some embodiments, the method includes contacting the biological sample with a combination of nucleases, such as a Uracil-Specific Excision Reagent (e.g., USER®) enzyme mix. In some aspects, one or more uracil residues can be incorporated into the non-complementary (single-stranded) region of the probe that includes the capture probe capture domain, such that the one or more uracils is found between the probe region hybridized to a target analyte and the non-complementary region of the probe. In some embodiments, a uracil-specific excision reagent can cleave the probe at the site of the one or more uracils, thereby releasing the capture probe capture domain of the probe. A uracil-specific excision reagent can comprise a uracil-DNA N-glycosylase (UNG or UDG), which catalyzes the excision of a uracil base, forming an abasic (apyrimidinic) site, while leaving the phosphodiester backbone intact. The uracil-specific excision reagent can further comprise an Endonuclease VIII. The lyase activity of Endonuclease VIII may break the phosphodiester backbone at the 3′ and 5′ sides of the abasic site to release the base-free deoxyribose. As such, the uracil-specific excision reagent may exhibit combined activities of a UNG and an Endonuclease VIII (e.g., a USER® enzyme). In some embodiments, the uracil-specific excision reagent catalyzes the excision of the one or more uracil residues. In some embodiments, the sample is contacted with 1 U, 2 U, 5 U, 10 U, 20 U, 30 U, 40 U, 50 U, 60 U, 70 U, 80 U, 90 U, or 100 U of a uracil-specific excision reagent. One unit (U) is defined as the amount of enzyme required to nick about 10 pmol of a 34-mer oligonucleotide duplex containing a single uracil base in 15 minutes at 37° C. in a total reaction volume of 10 μl.

In some embodiments, the method includes contacting the biological sample with a nuclease, such as a nickase. Nickases or nicking endonucleases can be used to hydrolyze only one strand of double-stranded DNA molecules to produce nicked DNA, rather than fully cleaved DNA. Like restriction endonucleases, nickases can recognize short, specific DNA recognition sequences and cleave the DNA strand at a fixed position relative to the recognition sequences. In some embodiments, the nickase recognizes a specific DNA recognition sequence of 1, 2, 3, 4, 5, 6, or more nucleotides in length. However, unlike most restriction endonucleases, nickases catalyze cleavage only one strand of a double-stranded polynucleotide. Non-limiting examples of nickases include: Nb.Bsml, Nb.Bts, Nt.Alwl, Nt.BbvC, Nt.BstNBI, and Nt.BpulOl. The lower-case letter “b” or “t” in the name of the nicking enzyme denotes whether the enzyme generates a nick in the bottom or top strand, respectively with the accepted linear convention being that the top strand runs from a free 5′ end on the left to a free 3′ end on the right, with the bottom strand in the opposite orientation. In some embodiments, the sample is contacted with at least any of 1 U, 2 U, 5 U, 10 U, 20 U, 30 U, 40 U, 50 U, 60 U, 70 U, 80 U, 90 U, or 100 U of a nickase. One unit is defined as the amount of enzyme, for instance Nt.BstNBI, required to digest 1 μg T7 DNA in 1 hour at 55° C. in a total reaction volume of 50 μl.

### Methods and Uses

Disclosed herein are methods of determining a location and/or abundance of a genetic variant such as a SNP in a nucleic acid in a biological sample. In some instances, the methods decrease non-specific binding of one or more probes to a target nucleic acid. In this instance, the non-specific binding of one or more probes to a target nucleic acid is decreased compared to methods that do not include contacting hybridized probes with an endonuclease.

As previously described, the methods disclosed herein can be performed on a single substrate that includes a spatial array having capture probes. In another embodiment, the methods can be performed on a system having two substrates in which templated ligation occurs on one substrate, and the ligation product is then captured by capture probes of a spatial array on a second substrate.

In some instances, the methods disclosed herein include contacting a first probe and a second probe with the biological sample on a first substrate, wherein the first probe and/or the second probe each includes one or more sequences complementary to a genetic variant in a target nucleic acid in the biological sample, and wherein the second probe includes a capture probe capture domain. The first probe and the second probe can each hybridize to the nucleic acid and be ligated together. In some embodiments, methods of targeted RNA capture provided herein include hybridizing a first probe and a second probe (e.g., a probe pair) to a target RNA. In some instances, the first and second probe each include sequences that are substantially complementary to one or more sequences (e.g., one or more target sequences) of the targeted RNA of interest. In some embodiments, the first probe and the second probe hybridize to complementary sequences that are adjacent (i.e., no gap of nucleotides) to one another on the same transcript. In some instances, there is a gap of nucleotides between the target RNA sequences. For instance, the first probe and the second probe hybridize to non-adjacent sequences that are at least 1, 2, 3, 4, 5, 6, 7, 8, 9, 10, or more nucleotides away from one another on the target RNA. In this instance, the methods include generating an extended first probe that includes a sequence substantially complementary to a sequence between the sequence hybridized to the first probe and the sequence hybridized to the second probe. This extension method can be performed using, e.g., a polymerase.

In some instances, the hybridization buffer includes saline-sodium citrate (SSC) (e.g., 1× SSC) or saline sodium phosphate EDTA (SSPE). In some instances, the hybridization buffer includes formamide or ethylene carbonate. In some instances, the hybridization buffer includes one or more salts, including but not limited to, a magnesium (Mg) salt, for example, MgCl_{2;} a sodium (Na) salt, for example, NaCl; or a manganese (Mn) salt, for example, MnCl₂. In some instances, the hybridization buffer includes Denhardt's solution, dextran sulfate, Ficoll®, PEG, or other hybridization rate accelerators. In some instances, the hybridization buffer includes a carrier such as yeast tRNA, salmon sperm DNA, and/or lambda phage DNA. In some instances, the hybridization buffer includes one or more blockers. In some instances, the hybridization buffer includes RNase inhibitor(s). In some instances, the hybridization buffer can include bovine serum albumin (BSA), sequence-specific blockers, non-specific blockers, EDTA, RNase inhibitor(s), betaine, TMAC, or DMSO. In some instances, a hybridization buffer can further include detergents such as Tween®, Triton-X® 100, sarkosyl, and SDS. In some instances, the hybridization buffer includes nuclease-free water or diethyl pyrocarbonate (DEPC) water.

In some instances, after hybridization, the biological sample is washed with a post-hybridization wash buffer. In some instances, the post-hybridization wash buffer includes SSC, yeast tRNA, formamide, ethylene carbonate, and/or nuclease-free water.

In some instances, a 5′ free end of the first probe and/or a 3′ free end of the second probe may be protected from nuclease digestion using blocking probes. In some instances, disclosed are blocking probes that hybridize to a capture probe capture domain. In some instances, the blocking probe and the capture probe capture domain are not on a continuous nucleotide sequence. That is, in some instances, each of the blocking probe and the capture probe capture domain on a first or second probe are independent nucleic acid sequences that hybridize together.

On the other hand, in some instances, the blocking probe and capture probe capture domain are on a contiguous nucleotide sequence and form a hairpin upon interaction. That is, in some instances, the blocking probe sequence is on a contiguous sequence with the first probe or the second probe. In some instances, the blocking probe and the 5′ free end of the first probe are on one contiguous nucleic acid sequence. In some instances, the blocking probe and the 3′ free end of the second probe are on one contiguous nucleic acid sequence. In some instances, the hairpin sequence includes a cleavable linker, such as, e.g., a photocleavable linker, UV-cleavable linker, or an enzyme-cleavable linker. In some instances, the hairpin sequence includes a target recognition sequence for a restriction endonuclease.

In some embodiments, the hairpin sequence is located 5′ (upstream) of the blocking probe. In some embodiments, the hairpin sequence is located 3′ (downstream) of the blocking probe. In some embodiments, the hairpin sequence is about 3 nucleotides, about 4 nucleotides, about 5 nucleotides, about 6 nucleotides, about 7 nucleotides, about 8 nucleotides, about 9 nucleotides, or about 10 or more nucleotides in length. In some instances, the hairpin sequence is at least about 15 nucleotides, at least about 20 nucleotides, at least about 25 nucleotides, at least about 30 nucleotides, or more nucleotides in length. In some embodiments, the hairpin sequence includes DNA, RNA, or modified nucleotides. The sequence of the hairpin (whether the sequence includes DNA, RNA, or modified nucleotides) can be nearly any nucleotide sequence so long as the sequence forms a hairpin, and in some instances, so long as the sequence is digestible by a nuclease of interest, e.g., USER.

In some instances, the blocking probe sequence is not on a contiguous sequence with the first probe or the second probe. In other words, in some instances, the first probe, the second probe, and the blocking sequence are three independent and separate sequences.

In some embodiments, the blocking probe includes a homopolymeric sequence that is substantially complementary to the capture probe capture domain. In some embodiments, the blocking probe is configured to hybridize to a poly(A), a poly(T), or a poly(U) sequence. In some embodiments, the blocking probe includes a poly(A), a poly(T), or a poly(U) sequence.

In some instances, the one or more blocking methods disclosed herein include use of caged nucleotides. In some embodiments, provided herein are methods where a capture probe capture domain includes a plurality of caged nucleotides. The caged nucleotides may be used to prevent the capture probe capture domain from interacting with the capture domain of the capture probe on a spatial array. The caged nucleotides may include caged moieties that block Watson-Crick hydrogen bonding, thereby preventing interaction until activation, for example, through photolysis of the caged moiety that releases the caged moiety and restores the caged nucleotide's ability to engage in Watson-Crick base pairing with a complementary nucleotide. Non-limiting examples of caged nucleotides are described in Liu et al., Acc. Chem. Res., 47(1): 45-55 (2014), which is incorporated by reference in its entirety. In some embodiments, the caged nucleotides include a caged moiety selected from the group consisting of 6-nitropiperonyloxymethyl (NPOM), 1-(ortho-nitrophenyl)-ethyl (NPE), 2-(ortho-nitrophenyl)-propyl (NPP), 7-(dicthylamino)-4-(hydroxymethyl)-coumarin (DEACM), and nitrodibenzofuran (NDBF).

In some embodiments, a caged nucleotide includes a non-naturally-occurring nucleotide selected from the group consisting of NPOM-caged adenosine, NPOM-caged guanosine, NPOM-caged uridine, and NPOM-caged thymidine. For example, the capture probe capture domain includes one or more NPOM-caged guanosines. In another example, the capture probe capture domain includes one or more NPOM-caged uridines. In yet another example, the capture probe capture domain includes one or more NPOM-caged thymidines. In some embodiments, the capture probe capture domain includes one caged nucleotide, two caged nucleotides, three caged nucleotides, four caged nucleotides, five caged nucleotides, six caged nucleotides, seven caged nucleotides, eight caged nucleotides, nine caged nucleotides, or ten or more caged nucleotides.

In some embodiments, after endonuclease treatment, the blocking probe is released. In some instances, releasing the blocking probe from the capture probe capture domain includes contacting the capture probe capture domain with a restriction endonuclease or an endoribonuclease. In some instances, releasing the blocking probe from the capture probe capture domain includes increasing the temperature of the biological sample. In some instances, releasing the blocking probe occurs substantially at the same time as releasing the ligation product from the target nucleic acid. In some instances, the releasing the blocking probe from the capture probe capture domain includes contacting the blocking probe with a UNG and an endonuclease. Non-limiting examples of endonucleases include one or more of Endonuclease VIII, Endonuclease III, Endonuclease V, uracil-DNA N-glycosylase 1 (UNG1), uracil-DNA N-glycosylase 2 (UNG2), single-strand-selective monofunctional uracil-DNA glycosylase 1 (SMUG1), thymine-DNA glycosylase (TDG), and methyl-CpG-binding domain-4 (MBD4).

When the blocking probe includes a hairpin sequence, releasing the blocking probe from the probe may include cleaving the hairpin sequence. In some embodiments, the hairpin sequence includes a cleavable linker. For example, the cleavable linker can be a photocleavable linker, UV-cleavable linker, chemical-cleavable linker, thermal-cleavable linker, or an enzyme-cleavable linker. In some embodiments, the enzyme that cleaves that enzymatic-cleavable domain is an endonuclease. In some embodiments, the hairpin sequence includes a target sequence for a restriction endonuclease.

In some embodiments, releasing the blocking probe from a blocked sequence (e.g., of a first or second probe) includes denaturing the blocking probe under conditions where the blocking probe de-hybridizes from the blocked sequence. In some embodiments, denaturing includes using chemical denaturation or physical denaturation. In some embodiments, denaturing includes temperature modulation. For example, a sequence and its blocking probe have predetermined annealing temperatures based on the nucleotide composition (A, G, C, or T) of the known sequences. In some embodiments, the temperature is modulated up by 5° C., up to 10° C., up to 15° C., up to 20° C., up to 25° C., up to 30° C., or up to 35° C. above the predetermined annealing temperature. In some embodiments, the temperature is modulated at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35° C. above the predetermined annealing temperature.

In some embodiments, the methods disclosed herein include releasing the caged moiety from the caged nucleotide. In some embodiments, releasing the caged moiety from the caged nucleotide includes activating the caged moiety. In some embodiments, releasing the caged moiety from the caged nucleotide restores the caged nucleotide's ability to hybridize to a complementary nucleotide through Watson-Crick hydrogen bonding. For example, restoring the caged nucleotide's ability to hybridize with a complementary nucleotide enables/restores the capture probe capture domain's ability to interact with a capture domain. Upon releasing the caged moiety from the caged nucleotide, the caged nucleotide is no longer “caged” in that the caged moiety is no longer linked (e.g., either covalently or non-covalently) to the caged nucleotide. As used herein, the term “caged nucleotide” can refer to a nucleotide that is linked to a caged moiety or a nucleotide that was linked to a caged moiety, but is no longer linked as a result of activation of the caged moiety.

In some embodiments, provided herein are methods for activating the caged moiety, thereby releasing the caged moiety from the caged nucleotide. In some embodiments, activating the caged moiety includes photolysis of the caged moiety from the nucleotide. As used herein, “photolysis” can refer to the process of removing or separating a caged moiety from a caged nucleotide using light. In some embodiments, activating the caged moiety (e.g., by photolysis) includes exposing the caged moiety to light pulses (e.g., two or more, three or more, four or more, or five or more pulses of light) that in total are sufficient to release the caged moiety from the caged nucleotide. In some embodiments, activating the caged moiety includes exposing the caged moiety to a light pulse (e.g., a single light pulse) that is sufficient to release the caged moiety from the caged nucleotide. In some embodiments, activating the caged moiety includes exposing the caged moiety to a plurality of light pulses (e.g., one, or two or more). In some embodiments, the light has a wavelength of about less than about 360 nm. In some embodiments, the source of the light that is at a wavelength of about less than 360 nm is a UV light. The UV light can originate from a fluorescence microscope, a UV laser or a UV flashlamp, or any source of UV light known in the art.

In some embodiments, when the caged moiety is released, the capture probe capture domain is able to hybridize to the capture domain of the capture probe on the spatial array.

The methods described herein also involve generating a ligation product by ligating the first probe and the second probe after the first probe and the second probe hybridize to their target analyte sequences in the biological sample. Ligation can be performed enzymatically or chemically. In some instances, the ligation is an enzymatic ligation reaction performed using a ligase (e.g., T4 RNA ligase (Rnl2), a PBCV-1 ligase, a single-stranded DNA ligase, or a T4 DNA ligase). See, e.g., Zhang et al.; RNA Biol. 2017; 14(1):36-44, which is incorporated by reference in its entirety, for a description of KOD ligase. In some embodiments, the ligase includes a thermostable 5′ App DNA/RNA ligase, a truncated T4 RNA ligase 2 (T4 Rnl2tr), a truncated T4 RNA ligase 2 K227Q, a truncated T4 RNA ligase 2 KQ, T4 RNA ligase R55K, K227Q, Chlorella Virus PBCV-1 DNA ligase, a Chlorella virus ligase, or a combination thereof.

Further, the methods include releasing the ligation product from the target analyte. The methods can include use of a reagent medium comprising a nuclease, a permeabilization agent, a detergent, or polyethylene glycol (PEG). In some instances, the nuclease includes an RNase. In some instances, the RNase is selected from RNase A, RNase C, RNase H, or RNase I.

The methods provided herein further include a permeabilization of the biological sample. In some embodiments, permeabilization is performed using a protease. In some embodiments, the protease is an endopeptidase. Endopeptidases that can be used include but are not limited to trypsin, chymotrypsin, elastase, thermolysin, pepsin, clostripan, glutamyl endopeptidase (Glu-C), Arg-C (clostripain), peptidyl-asp endopeptidase (ApsN), endopeptidase Lys-C and endopeptidase Lys-N. In some embodiments, the endopeptidase is pepsin. In some embodiments, after generating a ligation product, the biological sample is permeabilized. In some embodiments, the biological sample is permeabilized contemporaneously with or prior to contacting the biological sample with a first probe and a second probe, hybridizing the first probe and the second probe to the target analyte sequence, generating a ligation product by ligating the first probe and the second probe, and releasing the ligated product from the target analyte sequence.

In some embodiments, methods provided herein include permeabilization of the biological sample such that the capture probe on the array can more easily hybridize to the ligation product (i.e., compared to no permeabilization). In some embodiments, reverse transcription (RT) reagents can be added to permeabilized biological samples. Incubation with the RT reagents can produce spatially-barcoded extension products from the captured ligation products.

In some instances, permeabilization includes application of a permeabilization buffer to the biological sample. In some instances, the permeabilization buffer includes a buffering agent, such as Tris buffer (pH 7.5), MgCl₂, a sarkosyl detergent (e.g., sodium lauroyl sarcosinate), an enzyme (e.g., Proteinase K), and nuclease-free water. In some instances, permeabilization of a biological sample is performed at 37° C. In some instances, permeabilization is performed for about 20 minutes to about 2 hours (e.g., about 20 minutes, about 30 minutes, about 40 minutes, about 50 minutes, about 1 hour, about 1.5 hours, or about 2 hours). In some instances, permeabilization is performed for about 40 minutes.

In some embodiments, after generating a ligation product, the ligation product is released from the analyte. In some embodiments, a ligation product is released from the analyte using an endoribonuclease. In some embodiments, the endoribonuclease is RNase H, RNase A, RNase C, or RNase I. In some embodiments, the endoribonuclease is RNase H. RNase H is an endoribonuclease used to catalyze the hydrolysis of the phosphodiester bonds of RNA, when hybridized to DNA. RNase H is part of a conserved family of ribonucleases which are present in many different organisms. There are two primary classes of RNase H: RNase H1 and RNase H2. Retroviral RNase H enzymes are similar to the prokaryotic RNase H1. These enzymes share the characteristic ability to cleave the RNA component of an RNA: DNA heteroduplex. In some embodiments, the RNase H includes RNase H1, RNase H2, or both. In some embodiments, the RNase H includes but is not limited to RNase HII from Pyrococcus furiosus, RNase HII from Pyrococcus horikoshi, RNase HI from Thermococcus litoralis, RNase HI from Thermus thermophilus, RNAse HI from E. coli, or RNase HII from E. coli.

In some instances, the releasing of the ligation product from the analyte is performed using a releasing buffer. In some instances, the releasing buffer includes one or more of a buffering agent, such as Tris buffer (pH 7.5), an enzyme (e.g., RNAse H), and nuclease-free water. In some instances, the releasing is performed at 37° C. In some instances, the releasing is performed for about 20 minutes to about 2 hours (e.g., about 20 minutes, about 30 minutes, about 40 minutes, about 50 minutes, about 1 hour, about 1.5 hours, or about 2 hours). In some instances, the releasing is performed for about 30 minutes.

In some instances, the releasing of the ligation product occurs before permeabilization of the sample. In some instances, the releasing occurs after permeabilization. In some instances, the releasing occurs at the same time as permeabilization. In some instances, the ligation product hybridizes to a capture domain of a capture probe affixed to an array, wherein the array includes a plurality of capture probes, and wherein each capture probe of the plurality of capture probes includes: (i) a spatial barcode and (ii) a capture domain. In some instances, the capture probe further includes one or more functional domains, a unique molecular identifier (UMI), a cleavage domain, or a combination thereof. In some instances, the capture domain includes a homopolymeric sequence. In some instances, the capture domain includes a poly(T) sequence.

The methods disclosed herein also include determining sequences of (i) all or a part of the ligation product hybridized to the capture domain, or a complement thereof, and (ii) the spatial barcode, or a complement thereof, and using the determined sequence of (i) and (ii) to identify the location of the genetic variant in the biological sample or to determine an absence or presence of the genetic variant in the biological sample's transcriptome or genome. After a ligation product from the sample hybridizes or otherwise associates with a capture probe according to any of the methods described herein in connection with the general spatial cell-based analytical methodology, barcoded constructs or amplicons thereof that result from the hybridization/association may be analyzed.

For example, the capture probe can be extended (an “extended capture probe,” e.g., as described herein). Extending a capture probe can include generating a copy of the ligation product using the ligation product as a template and extension proceeds from a 3′ end of the capture domain where the ligation product is hybridized. Additionally, the ligation product can be extended to generate a molecule including the ligation product and a complement of the capture probe (e.g., using the capture probe as a template).

In some embodiments, the capture probe and/or ligation product is extended using a reverse transcriptase. In some embodiments, the capture probe and/or ligation product is extended using one or more DNA polymerases. Extension may occur in both directions, for example, the 3′ end of the capture probe may use the ligation product as a template to generate a copy of the ligation product on the capture probe affixed to the spatial array. Alternatively, or additionally, the 3′ end of the ligation product is extended using the capture probe as a template, thereby generating a ligation product that includes a complementary sequence of the capture probe, including a complement of the spatial barcode of the capture probe to which the ligation product is hybridized. In these instances, the ligation product extension product is not affixed to the spatial array, and as such, can be removed from the slide for further processing, such as library preparation for sequencing and determination of the presence or absence of a genetic variant. In some embodiments, extension is performed when the biological sample is present, whereas, in other embodiments, the biological sample is removed prior to extension of the capture probe and/or ligation product.

In some embodiments, extension products are amplified to yield quantities that are sufficient for analysis, e.g., via DNA sequencing. In some embodiments, the extended ligation product or complement or amplicon thereof is released and removed from the spatial array. The releasing of the extended ligation product or complement or amplicon thereof from the surface of the array (i.e., the capture probe on the array) can be achieved in a number of ways. In some embodiments, an extension product or a complement thereof is released from the array by denaturation (e.g., by heating to denature a double-stranded molecule or by chemical denaturation).

In some embodiments, detectable probes complementary to the extended capture probe can be contacted with the array to determine the presence or absence of a genetic variant. In some embodiments, the detectable probes can be labeled with a detectable label (e.g., a fluorescent, a chromogenic, or a chemiluminescent label). In some embodiments, detectable probes that do not bind (e.g., hybridize) to an extended capture probe can be washed away. In some embodiments, detectable probes complementary to the extended capture probe can be detected on the array (e.g., imaging, any of the detection methods described herein).

In some instances, the extended ligation products can be denatured from the extended capture probes and transferred off the array (e.g., to a clean tube) for amplification and/or library construction. The spatially-barcoded extended ligation products can be amplified via PCR prior to library construction. The amplicons can be enzymatically fragmented and size-selected in order to optimize for preferred amplicon size, for example, based on sequencing system requirements. For example, when using Illumina sequencing instruments P5 and P7 sequences directed to capturing the amplicons on a sequencing flowcell (Illumina sequencing instruments) can be appended to the amplicons, i7 and i5 can be used as sample indexes, and other sequencing related sequences can be added via End Repair, A-tailing, Adaptor Ligation, and PCR. The cDNA fragments can then be sequenced using paired-end sequencing using Read 1 and Read 2 sequences as sequencing primer sites. A skilled artisan will understand that additional or alternative sequences used by other sequencing instruments or technologies are also equally applicable for use in the aforementioned methods.

It is appreciated that the methods used herein can be combined with direct capture of RNA or detection of protein in the same sample where templated ligation to detect a genetic variant or mutation is performed. Methods of detection of RNA and protein are previously described and are incorporated herein.

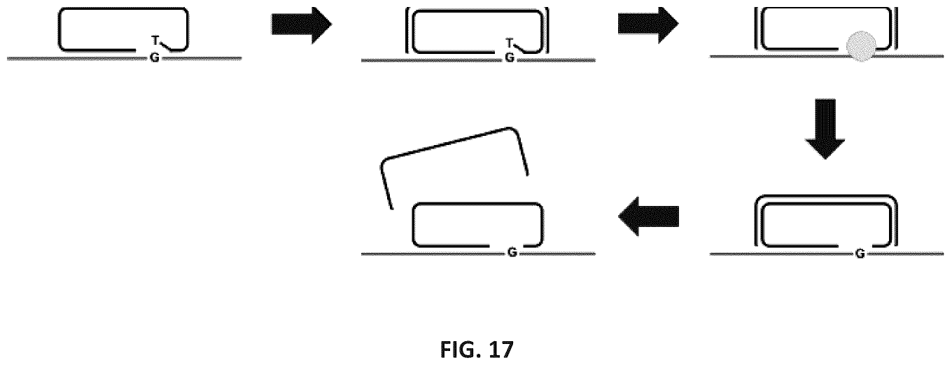

In some instances, the methods also include detecting a nucleic acid in a single cell or a single nucleus. The methods described herein include separating the biological sample into a plurality of partitions, each partition comprises a single cell or a single nucleus, wherein the plurality of partitions includes a plurality of gel beads, wherein a partition of the plurality of partitions comprises a gel bead of the plurality of gel beads, wherein a gel bead includes a capture probe having a cell barcode and a capture domain. After separating, a padlock probe can be hybridized to the nucleic acid as shown in FIG. 17. Then, a blocking oligonucleotide can hybridize to the padlock probe at a region outside of the sequences hybridized to the nucleic acid, i.e., the blocking oligonucleotide hybridizes to a sequence of the padlock probe that is not hybridized to the target nucleic acid. The blocking oligonucleotide can be used to protect the padlock probe from subsequent non-specific endonuclease cleavage. An endonuclease (e.g., S1 nuclease) can be contacted with the hybridized padlock probe, such that the nuclease cleaves the padlock probe at region including a mismatched nucleotide. After nuclease cleavage, the blocking oligonucleotide can be released from the padlock probe. The padlock probe can also be released from the target nucleic acid. The ordering of the releasing steps following nuclease cleavage can vary depending on the assay. The padlock probe can be circularized (e.g., by ligation) for downstream analysis. Any padlock probes that have undergone nuclease cleavage (i.e., contained a mismatch nucleotide), however, would be excluded from downstream analysis. After a circularized padlock probe is generated (e.g., via ligation) for those sequences that are not nicked/cleaved, all or part of the sequence of the padlock probe, or a complement thereof, and (ii) the sequence of the cell barcode, or a complement thereof, can be determined to detect the presence and spatial location nucleic acid in the biological sample. Determining the sequence (i.e., the presence of the SNP) can be performed via sequencing, as described herein. In some instances, the nucleic acid includes a SNP.

In some instances, the circularized padlock probe is amplified (e.g., using a polymerase). For instance, amplification can be performed using rolling circle amplification. In some instances, determining the presence and spatial location of the SNP can be performed by hybridizing a detectable probe to the ligation product; and detecting the detectable probe, which can have a fluorescent label or a chromogenic label.

### Compositions and Kits

In some embodiments, also provided herein are compositions, systems, and kits that include one or more reagents to detect one or more genetic variants in a biological sample. In some instances, a kit includes an arrayed substrate comprising a plurality of capture probes with each of the plurality of capture probes comprising a spatial barcode and a capture domain. In some instances, the kit includes a plurality of templated probes (e.g., a first probe, and a second probe) that can identify the presence or absence of a genetic variant or mutation.

A non-limiting example of a kit includes an array including a plurality of capture probes, wherein a capture probe of the plurality of capture probes includes: (i) a spatial barcode and (ii) a capture domain; a first probe and a second probe, wherein the first probe and the second probe each comprise a sequence that is substantially complementary to adjacent sequences of the nucleic acid, wherein the second probe includes a capture probe binding domain, and wherein the first probe and the second probe are capable of being ligated together to form a ligation product; and an endonuclease that cleaves a mismatched first probe and/or a mismatched second probe when hybridized to the nucleic acid, wherein the mismatched first probe differs from the first probe at the one or more sequences that are substantially complementary to sequences of the nucleic acid by at least one nucleotide, and instructions for performing the method any one of the methods disclosed herein.

A non-limiting example of a kit includes an array including a plurality of capture probes, wherein a capture probe of the plurality of capture probes includes: (i) a spatial barcode and (ii) a capture domain; a first probe and a second probe, wherein the first probe and the second probe each comprise a sequence that is substantially complementary to adjacent sequences of the target nucleic acid, wherein the second probe includes a capture probe capture domain complementary to a capture domain of a capture probe on the array, and wherein the first probe and the second probe are capable of being ligated together to form a ligation product; and an endonuclease that cleaves a mismatched first probe and/or a mismatched second probe when hybridized to the target nucleic acid, wherein the mismatched first probe differs from the first probe at the one or more sequences that are substantially complementary to sequences of the target nucleic acid by at least one nucleotide.

Compositions disclosed herein include compositions formed through any of the methods performed to determine the location and/or abundance of a SNP or mutation in a biological sample. The composition can include the components of the kit or system or any intermediate product formed during the methods in this disclosure.

In some instances, the system includes multiple substrates. The array can be on the first substrate or the second substrate. The system can include a support device configured to retain a first substrate and the second substrate, wherein the biological sample is placed on the first substrate. The system can include a second reagent medium for permeabilizing the biological sample. The system can include an alignment mechanism on the support device to align the first substrate and the second substrate.

### EXAMPLES

### Example 1: Methods of Identifying SNPs in a Target Nucleic Acid at a Location in a Biological Sample

As an overview, a non-limiting example of SNP detection using templated ligation on a biological sample (e.g., a tissue section) is performed. FFPE-fixed tissue samples are deparaffinized, stained (e.g., H&E stain), and imaged. Samples are destained (e.g., using HCl) and decrosslinked. Following decrosslinking, samples are treated with pre-hybridization buffer (e.g., hybridization buffer excluding the first and second probes). Then, first and second template-specific probes are added to the sample to allow hybridization to target nucleic acids in the sample, where one of the probes includes a sequence complementary to a SNP variant present in a target nucleic acid. Probes may hybridize to the target nucleic acid with complete or 100% complementarity (e.g., as shown in FIG. 12) or with one or more mismatches or less than 100% complementarity (e.g., as shown in FIG. 13A).

Blocking probes that are complementary to the 5′ end of the first probe and the 3′ end of the second probe are added to the sample. After hybridization of the first probe and the second probe to the target nucleic acid, and after hybridization of the blocking probes to the first and second probes, an S1 nuclease is added. S1 nuclease cleaves the first or second probe at any mismatches between the target nucleic acid and the first or second probe. Any first or second probes that are cleaved are thus decoupled from either the 5′ end of the first probe and the 3′ end of the second probe. The resultant ligation product of cleaved probes can be identified as such by downstream library preparation and sequencing. For example, if a first probe comprising a sequencing primer sequence (e.g., Read 2 sequence) is cleaved, the resulting ligation product of the cleaved first probe would lack the sequencing primer sequence. That resulting ligation product may be captured by a capture probe (e.g., if the cleaved first probe ligates to a second probe including a capture probe capture domain), but excluded in downstream library preparation. If a second probe including a capture probe capture domain (e.g., including a poly(A) sequence) is cleaved, the resulting ligation product of the cleaved second probe would lack the capture probe capture domain. That resulting ligation product would not be captured by a capture probe, and thus excluded in downstream library preparation. Samples are then washed to remove unhybridized probes.

Ligase is added to the samples to ligate hybridized templated probes to generate a ligation product and samples are washed again. Ligation products are released from the target nucleic acids by contacting the biological sample with RNase H. Samples are permeabilized to facilitate capture of the ligation products by the capture probes on the arrayed substrate. Captured ligation products and capture probes are extended. The extension products are denatured. Denatured, extended ligation products are removed from the substrate, sequencing libraries are generated, and the libraries are sequenced. Sequencing is used to determine the presence or absence of a SNP and its location in the biological sample based on the location in which the ligation products are captured by the capture probes on the arrayed substrate.

### Example 2: Methods of Identifying SNPs and Gene Expression in a Biological Sample

As an overview, a non-limiting example of SNP detection using templated ligation on a biological sample (e.g., a tissue section) is performed. FFPE-fixed tissue samples are deparaffinized, stained (e.g., H&E stain), and imaged. Samples are destained (e.g., using HCl) and decrosslinked. Following decrosslinking, samples are treated with pre-hybridization buffer (e.g., hybridization buffer excluding the first and second probes). Then, first and second template-specific probes are added to the sample to allow hybridization to their target nucleic acids in the sample, where one of the probes includes a sequence complementary to a SNP variant present in a target nucleic acid. Probes may hybridize to the target nucleic acid with complete or 100% complementarity (e.g., as shown in FIG. 12) or with one or more mismatches or less than 100% complementarity (e.g., as shown in FIG. 13A).

Blocking probes that are complementary to the 5′ end of the first probe and the 3′ end of the second probe are added to the sample. After hybridization of the first probe and the second probe to the target nucleic acid, and after hybridization of the blocking probes to the first and second probes, an S1 nuclease is added. S1 nuclease cleaves the first probe or the second probe at any mismatches between the target nucleic acid and the first or second probe. Samples are then washed to remove unhybridized probes.

Ligase is added to the samples to ligate hybridized templated probes to generate a ligation product and samples are washed again. Ligation products are released from the target nucleic acids by contacting the biological sample with RNase H. Samples are permeabilized to facilitate capture of the ligation products by the capture probes on the arrayed substrate. In this process, RNA molecules are also captured on the arrayed substrate, i.e., via polyadenylated end of the RNA molecules.

After hybridization of the RNA molecules and the ligation products, the capture probes are extended. The extension products are denatured. Denatured, extended ligation products are removed from the array, sequencing libraries are generated, and the libraries are sequenced. Sequencing is used to determine: (i) the presence or absence of a SNP and its location in the biological sample based on the location in which the ligation products are captured by the capture probes on the arrayed substrate and (ii) a whole transcriptomic gene expression profile based on the location in which the RNA molecules are captured by the capture probes on the arrayed substrate.

### Example 3: Methods of Identifying SNPs, Gene Expression, and Protein Expression in a Biological Sample

As an overview, a non-limiting example of SNP detection using templated ligation on a biological sample (e.g., a tissue section) is performed. FFPE-fixed tissue samples are deparaffinized, stained (e.g., H&E stain), and imaged. Samples are destained (e.g., using HCl) and decrosslinked. Following decrosslinking, samples are treated with pre-hybridization buffer (e.g., hybridization buffer excluding the first and second probes). Then, first and second template-specific probes are added to the sample to allow hybridization to their target nucleics acids in the sample, where one of the probes includes a sequence complementary to a SNP variant present in a target nucleic acid. Probes may hybridize to the target nucleic acid with complete or 100% complementarity (e.g., as shown in FIG. 12) or with one or more mismatches or less than 100% complementarity (e.g., as shown in FIG. 13A).

Blocking probes that are complementary to the 5′ end of the first probe and the 3′ end of the second probe are added to the sample. After hybridization of the first probe and the second probe to the target nucleic acid, and after hybridization of the blocking probes to the first and second probes, an S1 nuclease is added. S1 nuclease cleaves the first probe or the second probe at any mismatches between the target nucleic acid and the first or second probe. Samples are then washed to remove unhybridized probes.

Ligase is added to the samples to ligate hybridized templated probes to generate a ligation product and samples are washed. Ligation products are released from the target nucleic acids by contacting the biological sample with RNase H.

Following templated ligation probe hybridization, antibody-oligonucleotide conjugates (i.e., analyte capture agents) are incubated with the samples to allow the analyte binding moieties (e.g., antibodies) of the analyte capture agents to bind to protein targets (e.g., as described in PCT Patent Application Publication No. WO2021/133849A1). The analyte capture agents are added to the tissues, which are incubated in an antibody staining buffer.

Samples are permeabilized to facilitate capture of the ligation products by the capture probes on the arrayed substrate. In this process, the analyte capture agents are also captured on the arrayed substrate, i.e., via analyte capture sequences. After hybridization of the analyte capture agents and the ligation products, analyte capture agents, ligation products, and capture probes are extended. The extension products are denatured. Denatured, extended ligation products are removed from the array, sequencing libraries are generated, and the libraries are sequenced. Sequencing is used to determine: (i) the presence or absence of a SNP and its location in the biological sample based on the location in which the ligation products are captured by the capture probes on the arrayed substrate and (ii) a protein expression profile based on the location in which the analyte capture agents are captured by the capture probes on the arrayed substrate.

1. A method for spatially tagging a target nucleic acid in a biological sample, the method comprising:
(a) contacting a plurality of first probes and a plurality of second probes with the biological sample on a first substrate,
wherein the biological sample comprises a plurality of target nucleic acids, each target nucleic acid comprising either a wild-type sequence or a genetic variant sequence,
wherein each probe of the plurality of first probes and each probe of the plurality of second probes comprises a sequence complementary to a target nucleic acid of the plurality of target nucleic acids, and
wherein each probe of the plurality of first probes or each probe of the plurality of second probes comprises a capture probe capture domain that is complementary to a capture domain;
(b) hybridizing the plurality of first probes and the plurality of second probes to the plurality of target nucleic acids, thereby generating hybridized first and second probes, wherein the plurality of first probes and the plurality of second probes are configured to hybridize to both the wild-type sequence and the genetic variant sequence;
(c) contacting one or more blocking probes with the hybridized first and second probes,
wherein the one or more blocking probes are configured to hybridize to the hybridized first and second probes at regions that are not hybridized to the target nucleic acid, thereby generating protected first and second probes;
(d) contacting an endonuclease with the protected first and second probes, wherein the endonuclease cleaves protected first or second probes that are hybridized to target nucleic acids and include a mismatch;
(e) generating ligation products by ligating the protected first and second probes;
(f) releasing the ligation products from the target nucleic acids;
(g) releasing the one or more blocking probes from the ligation products; and
(h) hybridizing the ligation products to a plurality of capture probes affixed to an array,
wherein each capture probe of the plurality of capture probes comprises: (i) a spatial barcode and (ii) the capture domain, thereby spatially tagging the target nucleic acid in the biological sample.

2. (canceled)

3. The method of claim 1, wherein a probe of the plurality of first probes or of the plurality of second probes comprises a sequence complementary to the genetic variant sequence.

4-8. (canceled)

9. The method of claim 1, wherein the endonuclease cleaves the protected first and/or second probes or the protected ligation products that are hybridized to the genetic variant sequence that includes the mismatch.

10. The method of claim 1, wherein the endonuclease cleaves the protected first and/or second probes or the protected ligation products that are hybridized to the wild-type sequence that includes the mismatch.

11. The method of claim 1, wherein the endonuclease is an S1 nuclease.

12. (canceled)

13. The method of claim 1, wherein the one or more blocking probes comprises one or more sequences complementary to the 5′ single-stranded ends of the hybridized first probes and/or the 3′ single-stranded ends of the hybridized second probes.

14. The method of claim 1, wherein the one or more blocking probes comprises:
a poly(T) sequence;
a hairpin sequence located at 5′ ends of the one or more blocking probes;
a cleavable linker selected from the group consisting of a photocleavable linker, UV-cleavable linker, and an enzyme-cleavable linker; and/or
a target recognition sequence for a restriction endonuclease or an endoribonuclease.

15-19. (canceled)

20. The method of claim 1, wherein the releasing the one or more blocking probes comprises:
contacting the one or more blocking probes with a restriction endonuclease or an endoribonuclease;
heating the one or more blocking probes;
contacting the one or more blocking probes with a uracil-DNA glycosylase and a second endonuclease; and/or
photolysis.

21-29. (canceled)

30. The method of claim 1, wherein generating the ligation products comprises ligating hybridized first and second probes by enzymatic ligation, wherein the enzymatic ligation is performed using a ligase.

31-34. (canceled)

35. The method of claim 1, wherein the releasing the ligation products comprises contacting the ligation products with a nuclease.

36. (canceled)

37. The method of claim 1, wherein the releasing the ligation products from the target nucleic acids comprises using a reagent medium comprising a permeabilization agent, optionally wherein the permeabilization agent comprises a protease.

38-47. (canceled)

48. The method of claim 1, further comprising determining sequences of: (i) all or a part of the hybridized ligation products or a complement thereof, and (ii) the spatial barcodes or a complement thereof, and using the determined sequences of (i) and (ii) to identify a spatial location of the target nucleic acid in the biological sample.

49-52. (canceled)

53. The method of claim 1, wherein the plurality of first probes and the plurality of second probes hybridize to adjacent sequences on the target nucleic acids.

54. The method of claim 1, wherein the plurality of first probes and the plurality of second probes hybridize to non-adjacent sequences that are at least 1, 2, 3, 4, 5, 6, 7, 8, 9, 10, or more nucleotides away from one another on the target nucleic acids.

55. The method of claim 54, further comprising:
generating extended first probes, wherein the extended first probes comprise a sequence substantially complementary to a sequence between the sequence hybridized to the first probes and the sequence hybridized to the second probes; or
generating extended second probes using a polymerase, wherein the extended second probes comprise a sequence substantially complementary to a sequence between the sequence hybridized to the first probes and the sequence hybridized to the second probes.

56-58. (canceled)

59. The method of claim 1, wherein each capture probe further comprises one or more functional domains, a unique molecular identifier (UMI), a cleavage domain, or a combination thereof.

60-61. (canceled)

62. The method of claim 1, wherein the target nucleic acids are RNA or DNA.

63-65. (canceled)

66. The method of claim 1, wherein the first substrate comprises the array.

67. The method of claim 1, wherein the array is on a second substrate and the method further comprises aligning the first substrate with the second substrate such that at least a portion of the biological sample is aligned with at least a portion of the plurality of capture probes.

68. (canceled)

69. The method of claim 1, wherein the biological sample is a tissue sample.

70-148. (canceled)
